SEQUENCE LISTING (15 sequences)

SEQ ID NO: 1 - protein, 7 aa, Artificial Sequence
  CDRH1
SYNAVWN

SEQ ID NO: 2 - protein, 18 aa, Artificial Sequence
  CDRH2
RTYYRSGWYNDYAESVKS

SEQ ID NO: 3 - protein, 16 aa, Artificial Sequence
  CDRH3
SGHITVFGVNVDAFDM

SEQ ID NO: 4 - protein, 11 aa, Artificial Sequence
  CDRL1
RTSQSLSSYTH

SEQ ID NO: 5 - protein, 7 aa, Artificial Sequence
  CDRL2
AASSRGS

SEQ ID NO: 6 - protein, 5 aa, Artificial Sequence
  CDRL3
QQSRT

SEQ ID NO: 7 - protein, 128 aa, Artificial Sequence
  VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 8 - protein, 103 aa, Artificial Sequence
  VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 9 - protein, 458 aa, Artificial Sequence
  FluAB_MLNS heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 10 - protein, 210 aa, Artificial Sequence
  light chain
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 458 aa, Artificial Sequence
  FluAB_wt heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 384 bp, Artificial Sequence
  FluAB_MLNS VH nuc
caagttcagctgcagcagagcggccccggtctggtgaagcctagccagactctgtctttaacttgcgccatctccggcgacagcgtgagcagctacaacgccgtctggaactggattcgtcagagccctagcagaggtttagagtggctgggtcgtacttactatcgttccggctggtacaacgactacgccgagagcgtgaagtctcgtatcactatcaaccccgatactagcaagaaccagttctctttacagctgaacagcgtgactcccgaagacactgccgtgtactactgcgctcgtagcggccacatcactgtgttcggcgtgaatgtggacgccttcgacatgtggggccaaggtactatggtcactgtgagcagc

SEQ ID NO: 13 - DNA, 309 bp, Artificial Sequence
  FluAB_MLNS VL nuc
gacatccagatgactcagagcccttcctctttaagcgctagcgtgggcgatagggtcactatcacttgtcgtactagccagtctttaagctcctacactcactggtaccagcagaagcccggtaaggcccctaagctgctgatctacgctgccagcagcagaggcagcggagtgcctagcagatttagcggcagcggtagcggcactgacttcactctgacaatcagctctttacagcccgaagacttcgccacttactactgccagcagtctcgtactttcggccaaggtactaaggtggagatcaag

SEQ ID NO: 14 - DNA, 1374 bp, Artificial Sequence
  FluAB_MLNS heavy chain nuc
caagttcagctgcagcagagcggccccggtctggtgaagcctagccagactctgtctttaacttgcgccatctccggcgacagcgtgagcagctacaacgccgtctggaactggattcgtcagagccctagcagaggtttagagtggctgggtcgtacttactatcgttccggctggtacaacgactacgccgagagcgtgaagtctcgtatcactatcaaccccgatactagcaagaaccagttctctttacagctgaacagcgtgactcccgaagacactgccgtgtactactgcgctcgtagcggccacatcactgtgttcggcgtgaatgtggacgccttcgacatgtggggccaaggtactatggtcactgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaa

SEQ ID NO: 15 - DNA, 630 bp, Artificial Sequence
  FluAB_MLNS light chain nuc
gacatccagatgactcagagcccttcctctttaagcgctagcgtgggcgatagggtcactatcacttgtcgtactagccagtctttaagctcctacactcactggtaccagcagaagcccggtaaggcccctaagctgctgatctacgctgccagcagcagaggcagcggagtgcctagcagatttagcggcagcggtagcggcactgacttcactctgacaatcagctctttacagcccgaagacttcgccacttactactgccagcagtctcgtactttcggccaaggtactaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2022/0226470 A1
(43) Pub. Date: Jul. 21, 2022

(54) ANTIBODIES AND METHODS FOR TREATMENT OF INFLUENZA A INFECTION

(71) Applicant: HUMABS BIOMED SA, Bellinzona (CH)

(72) Inventors: Davide CORTI, Bellinzona (CH); Fabio BENIGNI, Bellinzona (CH)

(21) Appl. No.: 17/607,383

(22) PCT Filed: Apr. 30, 2020
(86) PCT No.: PCT/EP2020/062160

§ 371 Date: Oct. 28, 2021

(30) Foreign Application Priority Data
Apr. 30, 2019 (EP) PCT/EP2019/061134

(51) Int. Cl. A61K 39/42; C07K 16/10; A61P 31/16; A61K 45/06

(52) U.S. Cl. CPC A61K 39/42; C07K 16/1018; A61K 2039/545; A61K 45/06; A61P 31/16

(57) ABSTRACT

The present invention provides antibodies that neutralize infection of influenza A virus. The invention also provides nucleic acids that encode and immortalized B cells and cultured plasma cells that produce such antibodies. In addition, the invention provides the use of the antibodies of the invention in prophylaxis and treatment influenza A infection.

The invention relates to antibodies that potently reduce influenza A infection and to the use of such antibodies. In particular, the invention relates to the prophylaxis and treatment of influenza A infection.

Influenza is an infectious disease, which spreads around the world in yearly outbreaks resulting per year in about three to five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually the symptoms begin about two days after exposure to the virus. Complications of influenza may include pneumonia, sinus infections, and worsening of previous health problems such as asthma or heart failure, sepsis or exacerbation of chronic underling diseases.

Influenza is caused by influenza virus, an antigenically and genetically diverse group of viruses of the family Orthomyxoviridae that contains a negative-sense, single-stranded, segmented RNA genome. Of the four types of influenza virus (A, B, C and D), three types (A, B and C) affect humans. Influenza type A viruses are the most virulent human pathogens and cause the severest disease. Influenza A viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 contains H1, H2, H5, H6, H8, H9, H11, H12, H13, H16 and H17 subtypes, and group 2 includes H3, H4, H7, H10, H14 and H15 subtypes. While all subtypes are present in birds, mostly H1, H2 and H3 subtypes cause disease in humans. H5, H7 and H9 subtypes are causing sporadic severe infections in humans and may generate a new pandemic. Influenza A viruses continuously evolve generating new variants, a phenomenon called antigenic drift. As a consequence, antibodies produced in response to past viruses are poorly- or non-protective against new drifted viruses. A consequence is that a new vaccine has to be produced every year against H1 and H3 viruses that are predicted to emerge, a process that is very costly as well as not always efficient. The same applies to the production of a H5 influenza vaccine.

HA is a major surface protein of influenza A virus, which is the main target of neutralizing antibodies that are induced by infection or vaccination. HA is responsible for binding the virus to cells with sialic acid on the membranes, such as cells in the upper respiratory tract or erythrocytes. In addition, HA mediates the fusion of the viral envelope with the endosome membrane, after the pH has been reduced. HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha helical coiled coil structure and primarily forms the “stem” or “stalk” region of HA, while the HA1 region is a small globular domain containing a mix of a/p structures (“head” region of HA). The globular HA head region mediates binding to the sialic acid receptor, while the HA stem mediates the subsequent fusion between the viral and cellular membranes that is triggered in endosomes by the low pH. While the immunodominant HA globular head domain has high plasticity with distinct antigenic sites undergoing constant antigenic drift, the HA stem region is relatively conserved among subtypes. Current influenza vaccines mostly induce an immune response against the immunodominant and variable HA head region, which evolves faster than the stem region of HA (Kirkpatrick E, Qiu X, Wilson P C, Bahl J, Krammer F. The influenza virus hemagglutinin head evolves faster than the stalk domain. Sci Rep. 2018 Jul. 11; 8(1):10432). Therefore, a particular influenza vaccine usually confers protection for no more than a few years and annual re-development of influenza vaccines is required.

To overcome these problems, recently a new class of influenza-neutralizing antibodies that target conserved sites in the HA stem were developed as influenza virus therapeutics. These antibodies targeting the stem region of HA are usually broader neutralizing compared to antibodies targeting the head region of HA. An overview over broadly neutralizing influenza A antibodies is provided in Corti D. and Lanzavecchia A., Broadly neutralizing antiviral antibodies. Annu. Rev. Immunol. 2013; 31:705-742. Okuno et al. immunized mice with influenza virus A/Okuda/57 (H2N2) and isolated a monoclonal antibody (C179) that binds to a conserved conformational epitope in HA2 and neutralizes the Group 1 H2, H1 and H5 subtype influenza A viruses in vitro and in vivo in animal models (Okuno et al., 1993; Smirnov et al., 1999; Smirnov et al., 2000). Further examples of HA-stem region targeting antibodies include CR6261 (Throsby M, van den Brink E, Jongeneelen M, Poon L L M, Alard P, Cornelissen L, et al. (2008) Heterosubtypic Neutralizing Monoclonal Antibodies Cross-Protective against H5N1 and H1N1 Recovered from Human IgM⁺ Memory B Cells. PLoS ONE 3(12): e3942. https://doi.org/10.1371/journal.pone.0003942; Friesen R H E, Koudstaal W, Koldijk M H, Weverling G J, Brakenhoff J P J, Lenting P J, et al. (2010) New Class of Monoclonal Antibodies against Severe Influenza: Prophylactic and Therapeutic Efficacy in Ferrets. PLoS ONE 5(2): e9106. https://doi.org/10.1371/journal.pone.0009106), F10 (Sui J, Hwang W C, Perez S, Wei G, Aird D, Chen L M, Santelli E, Stec B, Cadwell G, Ali M, Wan H, Murakami A, Yammanuru A, Han T, Cox N J, Bankston L A, Donis R O, Liddington R C, Marasco W A (March 2009). “Structural and functional bases for broad-spectrum neutralization of avian and human influenza A viruses”. Nature Structural & Molecular Biology. 16 (3): 265-73. doi:10.1038/nsmb.1566), CR8020 (Ekiert D C, Friesen R H E, Bhabha G, Kwaks T, Jongeneelen M, et al. 2011. A highly conserved neutralizing epitope on group 2 influenza A viruses. Science 333(6044):843-50), F16 (Corti D, Voss J, Gamblin S J, Codoni G, Macagno A, et al. 2011. A neutralizing antibody selected from plasma cells that binds to group 1 and group 2 influenza A hemagglutinins. Science 333(6044):850-56), and CR9114 (Dreyfus C, Laursen N S, Kwaks T, Zuijdgeest D, Khayat R, et al. 2012. Highly conserved protective epitopes on influenza B viruses. Science 337(6100):1343-48).

However, antibodies capable of reacting with the HA stem region of both group 1 and 2 subtypes are extremely rare and usually do not show complete coverage of all subtypes. Recently, antibody MEDI8852 was described, which potently neutralizes group 1 and 2 influenza A viruses with unprecedented breadth, being able to neutralize a diverse panel of representative viruses spanning >80 years of antigenic evolution (Kallewaard N L, Corti D, Collins P J, et al. Structure and Function Analysis of an Antibody Recognizing All Influenza A Subtypes. Cell. 2016; 166(3):596-608; Paules, C. I. et al. The Hemagglutinin A Stem Antibody MEDI8852 Prevents and Controls Disease and Limits Transmission of Pandemic Influenza Viruses. J Infect Dis 216, 356-365, https://doi.org/10.1093/infdis/jix292 (2017)). MEDI8852 was shown to bind to a highly conserved epitope that is markedly different from other structurally characterized stem-reactive neutralizing antibodies (Kallewaard N L, Corti D, Collins P J, et al. Structure and Function Analysis of an Antibody Recognizing All Influenza A Subtypes. Cell. 2016; 166(3):596-608).

In view of the above, it is the object of the present invention to provide a novel antibody, which broadly and efficiently neutralizes influenza A virus, even when administered at very low doses.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “specifically binding” and similar reference does not encompass non-specific sticking.

As used herein, the term “antibody” encompasses various forms of antibodies including, without being limited to, whole antibodies, antibody fragments, human antibodies, chimeric antibodies, humanized antibodies, recombinant antibodies and genetically engineered antibodies (variant or mutant antibodies) as long as the characteristic properties according to the invention are retained. In some embodiments, the antibody is a human antibody. In some embodiments, the antibody is a monoclonal antibody. For example, the antibody is a human monoclonal antibody.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence.

A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Antibodies

The invention is based, amongst other findings, on the identification of antibodies that potently reduce influenza A infection even when administered at very low doses. In addition, the antibodies of the invention show an increased half-life. Without being bound to any theory, the present inventors assume that the increased potency of the antibody of the present invention is independent from the increased half-life. For example, in comparison to a comparative antibody, the antibody of the invention showed increased potency despite similar plasma concentrations of the antibody. Moreover, the antibodies of the present invention surprisingly show decreased immunogenicity as compared to parental antibodies without the mutations M428L and N434S in the constant region of the heavy chain.

In a first aspect the present invention provides an (isolated) antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S in the constant region of the heavy chain.

In general, the antibody according to the present invention, typically comprises (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain), there are six CDRs for each antigen receptor (heavy chain: CDRH1, CDRH2, and CDRH3; light chain: CDRL1, CDRL2, and CDRL3). A single antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDR's.

The sequences of the heavy chains and light chains of exemplary antibodies of the invention, comprising three different CDRs on the heavy chain and three different CDRs on the light chain were determined. The position of the CDR amino acids are defined according to the IMGT numbering system (IMGT: http://www.imgt.org/; cf. Lefranc, M.-P. et al. (2009) Nucleic Acids Res. 37, D1006-D1012).

Typically, the antibody of the invention binds to hemagglutinin of an influenza A virus. Thereby, the antibody of the invention can neutralize infection of influenza A virus. By virtue of the six CDR sequences as defined above, the antibody according to the present invention binds to the same epitope of the influenza A virus hemagglutinin (IAV HA) stem region as MEDI8852 (Kallewaard N L, Corti D, Collins P J, et al. Structure and Function Analysis of an Antibody Recognizing All Influenza A Subtypes. Cell. 2016; 166(3):596-608), thereby providing the same broad protection against various influenza A serotypes of all influenza A subtypes.

In addition, the antibody of the present invention includes two mutations in the constant region of the heavy chain (in the CH3 region): M428L and N434S. In this context, the amino acid positions have been numbered according to the art-recognized EU numbering system. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest”, 5^{th} edition, Bethesda, Md.: U.S. Dept. of Health and Human Services, Public Health Service, National Institutes of Health, 1991, hereby entirely incorporated by reference).

In some embodiments, the antibody of the invention neutralizes influenza A infection at a dose, which does not exceed half of the dose required for neutralization of influenza A with a comparative antibody, which differs from said antibody only in that it does not contain the mutations M428L and N434S in the constant region of the heavy chain. In some embodiments, the dose of the antibody of the invention does not exceed one third of the dose required for neutralization of influenza A with said comparative antibody. In some embodiments, the dose of the antibody of the invention does not exceed one quarter of the dose required for neutralization of influenza A with said comparative antibody. In some embodiments, the dose of the antibody of the invention does not exceed one fifth of the dose required for neutralization of influenza A with said comparative antibody. In some embodiments, the dose of the antibody of the invention does not exceed one sixth of the dose required for neutralization of influenza A with said comparative antibody. In some embodiments, the dose of the antibody of the invention does not exceed one seventh of the dose required for neutralization of influenza A with said comparative antibody. In some embodiments, the dose of the antibody of the invention does not exceed one eighth of the dose required for neutralization of influenza A with said comparative antibody. In some embodiments, the dose of the antibody of the invention does not exceed one ninth of the dose required for neutralization of influenza A with said comparative antibody. In some embodiments, the dose of the antibody of the invention does not exceed one tenth of the dose required for neutralization of influenza A with said comparative antibody. It is understood that for such comparative tests comparable neutralization assays are used (similar test assays, test conditions etc.). For example, the same test (differing only in the antibodies to be tested) may be used to determine the dose for the antibody of the invention for neutralization of influenza A and for determining the dose for the comparative antibody for neutralization of influenza A.

To study and quantitate virus infectivity (or “neutralization”) in the laboratory the person skilled in the art knows various standard “neutralization assays”. For a neutralization assay animal viruses are typically propagated in cells and/or cell lines. For example, in a neutralization assay cultured cells may be incubated with a fixed amount of influenza A virus (IAV) in the presence (or absence) of the antibody to be tested. As a readout for example flow cytometry may be used. Alternatively, also other readouts are conceivable.

In certain embodiments, the antibody neutralizes viruses encoding polymorphisms HA1 P11S, HA2 D46N, and/or HA2 N49T of H3N2 hemagglutinin (H3 HA); and/or polymorphism N146D of H1N1 hemagglutinin (H1 HA). For example, the antibody may neutralize one or two polymorphisms of HA1 P11S, HA2 D46N or HA2 N49T of H3 HA. In particular, the antibody may neutralize all three polymorphisms HA1 P11S, HA2 D46N, and HA2 N49T of H3 HA. Moreover, the antibody may neutralize polymorphism N146D of H1 HA. In some embodiments, the antibody neutralizes polymorphisms HA1 P11S, HA2 D46N, and HA2 N49T of H3 HA; and polymorphism N146D of H1 HA. For said polymorphisms, the reference for H1N1 is A/California/07/2009 and the reference for H3N2 is A/Perth/16/2009.

In certain instances, the antibody neutralizes polymorphisms HA1 P11S, HA2 D46N, and/or HA2 N49T of H3 HA; and/or polymorphism N146D of H1 HA with IC₅₀ fold changes of <2 relative to HA of the wild type virus, in particular in a side-by-side comparison with the wild type virus. For example, the antibody may neutralize one or two polymorphisms of HA1 P11S, HA2 D46N or HA2 N49T of H3 HA with IC₅₀ fold changes of <2 relative to HA of the wild type virus, in particular in a side-by-side comparison with the wild type virus. In particular, the antibody may neutralize all three polymorphisms HA1 P11S, HA2 D46N, and HA2 N49T of H3 HA with IC₅₀ fold changes of <2 relative to HA of the wild type virus, in particular in a side-by-side comparison with the wild type virus. Moreover, the antibody may neutralize polymorphism N146D of H1 HA with IC₅₀ fold changes of <2 relative to HA of the wild type virus, in particular in a side-by-side comparison with the wild type virus. In some embodiments, the antibody neutralizes polymorphisms HA1 P11S, HA2 D46N, and HA2 N49T of H3 HA; and polymorphism N146D of H1 HA, each with IC₅₀ fold changes of <2 relative to HA of the wild type virus, in particular in a side-by-side comparison with the wild type virus.

In some embodiments, the antibody elicits a decreased anti-drug antibody (ADA) response as compared to a comparative antibody differing from said antibody only in that it does not contain the mutations M428L and N434S in the constant region of the heavy chain. In particular, the antibody may exhibit less immunogenicity as compared to a comparative antibody differing from said antibody only in that it does not contain the mutations M428L and N434S in the constant region of the heavy chain. As shown in the examples of the present specification, the antibody of the invention surprisingly elicits a decreased anti-drug antibody (ADA) response and, thus, less immunogenicity as compared to an antibody without the M428L/N434S mutations. For assessing anti-drug antibody (ADA) responses/immunogenicity, the skilled person is aware of appropriate tests. Any of such tests may be selected as long as the antibody of the invention and the comparative antibody without the M428L/N434S mutations are test side by side to enable direct comparison. Exemplified tests are described in examples 9 and 10 of the present specification.

In some embodiments, the antibody of the invention is a human antibody. In some embodiments, the antibody of the invention is a monoclonal antibody. For example, the antibody of the invention is a human monoclonal antibody.

Antibodies of the invention can be of any isotype (e.g., IgA, IgG, IgM i.e. an x, y or Ip heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody has a kappa (κ) light chain. In some embodiments, the antibody is of IgG1 type and has a κ light chain.

In some embodiments, the antibody is of the human IgG1 type. The antibody may be of any allotype. The term “allotype” refers to the allelic variation found among the IgG subclasses. For example, the antibody may be of the G1m1 (or G1m(a)) allotype, of the G1m2 (or G1m(x)) allotype, of the G1m3 (or G1m(f)) allotype, and/or of the G1m17 (or Gm(z)) allotype. The G1m3 and G1m17 allotypes are located at the same position in the CHI domain (position 214 according to EU numbering). G1m3 corresponds to R214 (EU), while G1m17 corresponds to K214 (EU). The G1m1 allotype is located in the CH3 domain (at positions 356 and 358 (EU)) and refers to the replacements E356D and M358L. The G1m2 allotype refers to a replacement of the alanine in position 431 (EU) by a glycine. The G1m1 allotype may be combined, for example, with the G1m3 or the G1m17 allotype. In some embodiments, the antibody is of the allotype G1m3 with no G1m1 (G1m3, −1). In some embodiments, the antibody is of the G1m17,1 allotype. In some embodiments, the antibody is of the G1m3,1 allotype. In some embodiments, the antibody is of the allotype G1m17 with no G1m1 (G1m17, −1). Optionally, these allotypes may be combined (or not combined) with the G1m2, G1m27 or G1m28 allotype. For example, the antibody may be of the G1m17,1,2 allotype.

In some embodiments, the antibody of the invention comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 70% identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are maintained.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e. any combination of deletions, insertions or substitutions, per 100 amino acids of the reference sequence.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In some embodiments, the antibody of the invention comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 75% identity to SEQ ID NO: 8, wherein the CDR sequences as defined above are maintained. In some embodiments, the antibody of the invention comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 80% identity to SEQ ID NO: 8, wherein the CDR sequences as defined above are maintained. In some embodiments, the antibody of the invention comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 85% identity to SEQ ID NO: 8, wherein the CDR sequences as defined above are maintained. In some embodiments, the antibody of the invention comprises a heavy chain variable region comprising an amino acid sequence having 90% or more (i.e. 91%, 92%, 93%, 94%, 95%, 96%, 97%, 98%, 99% or more) identity to SEQ ID NO: 7 and a light chain variable region comprising the amino acid sequence having at least 90% identity to SEQ ID NO: 8, wherein the CDR sequences as defined above are maintained. In some embodiments, the antibody of the invention comprises a heavy chain variable region comprising an amino acid sequence having 95% or more (i.e. 96%, 97%, 98%, 99% or more) identity to SEQ ID NO: 7 and a light chain variable region comprising the amino acid sequence having at least 95% identity to SEQ ID NO: 8, wherein the CDR sequences as defined above are maintained.

In some embodiments, the antibody of the invention comprises a heavy chain variable region comprising an amino acid sequence as set forth in SEQ ID NO: 7 and a light chain variable region comprising the amino acid sequence as set forth in SEQ ID NO: 8, wherein the CDR sequences as defined above are maintained.

In general, it is possible that the antibody of the invention comprises one or more further mutations (in addition to M428L and N434S) in the Fc region (e.g., in the CH2 or CH3 region). However, in some embodiments, the antibody of the invention does not comprise any further mutation in addition to M428L and N434S in its CH3 region (in comparison to the respective wild-type CH3 region). In some embodiments, the antibody of the invention does not comprise any further mutation in addition to M428L and N434S in its Fc region (in comparison to the respective wild-type Fc region). As used herein, the term “wild-type” refers to the reference sequence, for example as occurring in nature. As a specific example, the term “wild-type” may refer to the sequence with the highest prevalence occurring in nature.

In some embodiments, the antibody of the invention comprises a heavy chain comprising an amino acid sequence as set forth in SEQ ID NO: 9 and a light chain comprising an amino acid sequence as set forth in SEQ ID NO: 10. For example, the antibody of the invention may have a heavy chain consisting of an amino acid sequence as set forth in SEQ ID NO: 9 and a light chain consisting of an amino acid sequence as set forth in SEQ ID NO: 10.

Antibodies of the invention also include hybrid antibody molecules that comprise the six CDRs from an antibody of the invention as defined above and one or more CDRs from another antibody to the same or a different epitope or antigen. In some embodiments, such hybrid antibodies comprise six CDRs from an antibody of the invention and six CDRs from another antibody to a different epitope or antigen.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

Nucleic Acids

In another aspect, the invention also provides a nucleic acid molecule comprising a polynucleotide encoding the antibody according to the present invention as described above.

In certain embodiments, the nucleic acid molecule comprises
(i) a polynucleotide comprising a nucleotide sequence as set forth in SEQ ID NO: 12; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 12, the nucleotide sequence encoding the CDR sequences as defined above; and
(ii) a polynucleotide comprising a nucleotide sequence as set forth in SEQ ID NO: 13; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13, the nucleotide sequence encoding the CDR sequences as defined above.

In some embodiments, the nucleic acid molecule comprises
(i) a polynucleotide comprising a nucleotide sequence as set forth in SEQ ID NO: 14; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 14, the nucleotide sequence encoding the CDR sequences as defined above and the M428L and N434S mutations in the constant region; and
(ii) a polynucleotide comprising a nucleotide sequence as set forth in SEQ ID NO: 15; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, the nucleotide sequence encoding the CDR sequences as defined above and the M428L and N434S mutations in the constant region.

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of the antibody of the invention. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. The exemplified nucleic acid sequences according to SEQ ID NOs 12, 13, 14 and 15 are codon-optimized sequences for the expression of exemplified antibody FluAB_MLNS. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody of the invention can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1).

The present invention also provides a combination of a first and a second nucleic acid molecule, wherein the first nucleic acid molecule comprises a polynucleotide encoding the heavy chain of the antibody of the present invention; and the second nucleic acid molecule comprises a polynucleotide encoding the corresponding light chain of the same antibody. The above description regarding the (general) features of the nucleic acid molecule of the invention applies accordingly to the first and second nucleic acid molecule of the combination. For example, one or both of the polynucleotides encoding the heavy and/or light chain(s) of the antibody, or an antigen-binding fragment thereof, may be codon-optimized.

In certain embodiments, the combination of nucleic acid molecules comprises
(i) a first nucleic acid molecule comprising a polynucleotide encoding the heavy chain of an antibody, the polynucleotide comprising a nucleotide sequence as set forth in SEQ ID NO: 12; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 12, the nucleotide sequence encoding the CDR sequences as defined above; and
(ii) a second nucleic acid molecule comprising a polynucleotide encoding the light chain of an antibody, the polynucleotide comprising a nucleotide sequence as set forth in SEQ ID NO: 13; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13, the nucleotide sequence encoding the CDR sequences as defined above.

In some embodiments, the combination of nucleic acid molecules comprises
(i) a first nucleic acid molecule comprising a polynucleotide encoding the heavy chain of an antibody, the polynucleotide comprising a nucleotide sequence as set forth in SEQ ID NO: 14; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 14, the nucleotide sequence encoding the CDR sequences as defined above and the M428L and N434S mutations in the constant region; and
(ii) a second nucleic acid molecule comprising a polynucleotide encoding the light chain of an antibody, the polynucleotide comprising a nucleotide sequence as set forth in SEQ ID NO: 15; or a nucleotide sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, the nucleotide sequence encoding the CDR sequences as defined above and the M428L and N434S mutations in the constant region.

Vector

Further included within the scope of the invention are vectors, for example, expression vectors, comprising a nucleic acid molecule according to the present invention or the combination of nucleic acid molecules according to the present invention (e.g., in bicistronic manner). Usually, a vector comprises a nucleic acid molecule as described above or a combination of nucleic acid molecules as described above (e.g., in bicistronic manner).

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

Cells

In a further aspect, the present invention also provides cell expressing the antibody according to the present invention; and/or comprising the vector according the present invention.

Examples of such cells include but are not limited to, eukaryotic cells, e.g., yeast cells, animal cells or plant cells or prokaryotic cells, including E. coli. In some embodiments, the cells are mammalian cells, such as a mammalian cell line. Examples include human cells, CHO cells, HEK293T cells, PER.C6 cells, NSO cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., reduced immunogenicity). Accordingly, the antibody of the invention, or the antigen-binding fragment thereof, may have a post-translational modification, which is distinct from the naturally produced antibody (e.g., an antibody of an immune response in a human).

Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the invention are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293T, PER.C6, NSO, myeloma or hybridoma cells. In other embodiments, the expression of the DNA sequence encoding the antibody molecules of the present invention to be used may be expressed in prokaryotic cells, including, but not limited to, E. coli.

The present invention also provides a process for the production of an antibody molecule according to the present invention comprising culturing a (heterologous) host cell comprising a vector encoding a nucleic acid of the present invention under conditions suitable for expression of protein from DNA encoding the antibody molecule of the present invention, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the invention can be used in various ways e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources may be more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the invention also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NSO cells, human cells such as PER.C6 or HKB-11 cells, myeloma cells, or a human liver cell), as well as plant cells. In some embodiments, the transfected host cell may a prokaryotic cell, including E. coli. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method for preparing one or more nucleic acid molecules (e.g., heavy and light chain genes) that encode an antibody of interest, comprising the steps of: (i) preparing an immortalized B cell clone or culturing plasma cells according to the invention; (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cells that encodes the antibody of interest.

The invention further provides a method of preparing nucleic acid molecule(s) that encode an antibody of interest, comprising the step of obtaining the nucleic acid that was obtained from a transformed B cell clone or cultured plasma cells of the invention. Thus the procedures for first obtaining the B cell clone or the cultured plasma cell, and then obtaining nucleic acid(s) from the B cell clone or the cultured plasma cells can be performed at different times by different people in different places (e.g., in different countries).

The invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

The present invention also provides a method of decreasing the immunogenicity of an antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; the method comprising a step of introducing the mutations M428L and N434S in the constant region of the heavy chain of the antibody. The mutations may be achieved as described above. As shown in the examples of the present specification, the antibody of the invention surprisingly exhibits very low immunogenicity only, in particular less immunogenicity as compared to the antibody without the M428L/N434S mutations. Accordingly, introducing those mutations into an antibody decreases immunogenicity of the antibody.

Pharmaceutical Composition

The present invention also provides a pharmaceutical composition comprising one or more of:
(i) the antibody according to the present invention;
(ii) the nucleic acid encoding the antibody according to the present invention;
(iii) the vector comprising the nucleic acid according to the present invention; and/or
(iv) the cell expressing the antibody according to the present invention or comprising the vector according to the present invention

and, optionally, a pharmaceutically acceptable diluent or carrier.

In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present invention is not an active component in respect to influenza A virus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Once formulated, the compositions of the invention can be administered directly to the subject. In some embodiments the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present invention that is to be given to an individual, administration is usually in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In some embodiments, the amount of the antibody in the pharmaceutical composition—in particular if provided as single-dose product—does not exceed 200 mg, for example it does not exceed 100 mg or 50 mg.

For example, the pharmaceutical composition according to the present invention may be administered daily, e.g. once or several times per day, e.g. once, twice, three times or four times per day, for 1, 2, 3, 4, 5, 6, 7, 8, 9, 10, 11, 12, 13, 14, 15, 16, 17, 18, 19, 20 or 21 or more days, e.g. daily for 1, 2, 3, 4, 5, 6 months. In some embodiments, the pharmaceutical composition according to the present invention may be administered weekly, e.g. once or twice per week, for 1, 2, 3, 4, 5, 6, 7, 8, 9, 10, 11, 12, 13, 14, 15, 16, 17, 18, 19, 20 or 21 or more weeks, e.g. weekly for 1, 2, 3, 4, 5, 6, 7, 8, 9, 10, 11, or 12 months or weekly for 2, 3, 4, or 5 years. Moreover, the pharmaceutical composition according to the present invention may be administered monthly, e.g. once per month or every second month for 1, 2, 3, 4, or 5 or more years. Administration may also continue for the lifetime. In some embodiments, one single administration only is also envisaged, in particular in respect to certain indications, e.g. for prophylaxis of influenza A virus infection. For example, a single administration (single dose) is administered and further doses may be administered at one or more later time points, when the titer of the antibody is insufficient or assumed to be insufficient for protection.

For a single dose, e.g. a daily, weekly or monthly dose, the amount of the antibody in the pharmaceutical composition according to the present invention, may not exceed 1 g or 500 mg. In some embodiments, for a single dose, the amount of the antibody in the pharmaceutical composition according to the present invention, may not exceed 200 mg, or 100 mg. For example, for a single dose, the amount of the antibody in the pharmaceutical composition according to the present invention, may not exceed 50 mg.

Pharmaceutical compositions typically include an “effective” amount of one or more antibodies of the invention, i.e. an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g. amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. For example, the pharmaceutical composition may comprise one or more antivirals (which are not antibodies). Moreover, the pharmaceutical composition may also comprise one or more antibodies (which are not according to the invention), for example an antibody against other influenza virus antigens (other than hemagglutinin) or an antibody against another influenza virus (e.g., against an influenza B virus or against an influenza C virus). Accordingly, the pharmaceutical composition according to the present invention may comprise one or more of the additional active components.

The antibody according to the present invention can be present either in the same pharmaceutical composition as the additional active component or, alternatively, the antibody according to the present invention is comprised by a first pharmaceutical composition and the additional active component is comprised by a second pharmaceutical composition different from the first pharmaceutical composition. Accordingly, if more than one additional active component is envisaged, each additional active component and the antibody according to the present invention may be comprised in a different pharmaceutical composition. Such different pharmaceutical compositions may be administered either combined/simultaneously or at separate times or at separate locations (e.g. separate parts of the body).

The antibody according to the present invention and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically-acceptable carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the invention.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In some embodiments, one or more of the immunoregulatory agents include(s) an adjuvant.

Medical Treatments and Uses

In a further aspect, the present invention provides the use of the antibody according to the present invention, the nucleic acid according to the present invention, the vector according to the present invention, the cell according to the present invention or the pharmaceutical composition according to the present invention in prophylaxis and/or treatment of infection with influenza A virus; or in (ii) diagnosis of infection with influenza A virus. Accordingly, the present invention also provides a method of reducing influenza A virus infection, or lowering the risk of influenza A virus infection, comprising: administering to a subject in need thereof, a therapeutically effective amount of the antibody according to the present invention, the nucleic acid according to the present invention, the vector according to the present invention, the cell according to the present invention or the pharmaceutical composition according to the present invention. Moreover, the present invention also provides the use of the antibody according to the present invention, the nucleic acid according to the present invention, the vector according to the present invention, the cell according to the present invention or the pharmaceutical composition according to the present invention in the manufacture of a medicament for prophylaxis, treatment or attenuation of influenza A virus infection.

Methods of diagnosis may include contacting an antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. The methods of diagnosis may also include the detection of an antigen/antibody complex, in particular following the contacting of an antibody with a sample. Such a detection step is typically performed at the bench, i.e. without any contact to the human or animal body. Examples of detection methods are well-known to the person skilled in the art and include, e.g., ELISA (enzyme-linked immunosorbent assay).

Prophylaxis of infection with influenza A virus refers in particular to prophylactic settings, wherein the subject was not diagnosed with infection with influenza A virus (either no diagnosis was performed or diagnosis results were negative) and/or the subject does not show symptoms of infection with influenza A virus. Prophylaxis of infection with influenza A virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza A virus is also particularly useful in subjects at greater risk acquiring influenza A virus infection, e.g. due to increased exposure, for example subjects working or staying in public areas, in particular health care workers.

In therapeutic settings, in contrast, the subject is typically infected with influenza A virus, diagnosed with influenza A virus infection and/or showing symptoms of influenza A virus infection. Of note, the terms “treatment” and “therapy”/“therapeutic” of influenza A virus infection include (complete) cure as well as attenuation/reduction of influenza A virus infection and/or related symptoms.

Accordingly, the antibody according to the present invention, the nucleic acid according to the present invention, the vector according to the present invention, the cell according to the present invention or the pharmaceutical composition according to the present invention may be used for treatment of influenza A virus infection in subjects diagnosed with influenza A virus infection or in subjects showing symptoms of influenza A virus infection.

The antibody according to the present invention, the nucleic acid according to the present invention, the vector according to the present invention, the cell according to the present invention or the pharmaceutical composition according to the present invention may also be used for prophylaxis and/or treatment of influenza A virus infection in asymptomatic subjects. Those subjects may be diagnosed or not diagnosed with influenza A virus infection.

In some embodiments, the subject to be treated (e.g., in prophylactic or therapeutic settings as described above) suffers from an autoimmune disease or an allergy; or is at risk of developing an autoimmune disease or an allergy. Subjects at risk of developing an autoimmune disease or an allergy include subjects having family members with autoimmune diseases and/or allergies, and subjects (regularly) exposed to allergens. As shown in the examples of the present specification, the antibody of the invention surprisingly exhibits very low immunogenicity only, in particular less immunogenicity as compared to the antibody without the M428L/N434S mutations. Accordingly, the antibody of the invention may be particularly useful in subjects at risk of extensive immune responses.

In some embodiments, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to three months before (a possible) influenza A virus infection or up to one month before (a possible) influenza A virus infection, such as up to two weeks before (a possible) influenza A virus infection or up to one week before (a possible) influenza A virus infection. For example, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to one day before (a possible) influenza A virus infection. Such a treatment schedule refers in particular to a prophylactic setting.

Moreover, the antibody according to the present invention, the nucleic acid according to the present invention, the vector according to the present invention, the cell according to the present invention or the pharmaceutical composition according to the present invention may be used for prophylaxis and/or treatment of influenza A virus infection, wherein the antibody, the nucleic acid, the vector, the cell, or the pharmaceutical composition is administered up to three months before the first symptoms of influenza A infection occur or up to one month before the first symptoms of influenza A infection occur, such as up to two weeks the first symptoms of influenza A infection occur or up to one week before the first symptoms of influenza A infection occur. For example, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to three days or two days before the first symptoms of influenza A infection occur.

In general after the first administration of the antibody according to the present invention, the nucleic acid according to the present invention, the vector according to the present invention, the cell according to the present invention or the pharmaceutical composition according to the present invention, one or more subsequent administrations may follow, for example a single dose per day or per every second day for 1, 2, 3, 4, 5, 6, 7, 8, 9, 10, 11, 12, 13, 1, 15, 16, 17, 18, 19, 20, or 21 days. After the first administration of the antibody according to the present invention, the nucleic acid according to the present invention, the vector according to the present invention, the cell according to the present invention or the pharmaceutical composition according to the present invention, one or more subsequent administrations may follow, for example a single dose once or twice per week for 1, 2, 3, 4, 5, 6, 7, 8, 9, 10, 11, 12, 13, 1, 15, 16, 17, 18, 19, 20, or 21 weeks. After the first administration of the antibody according to the present invention, the nucleic acid according to the present invention, the vector according to the present invention, the cell according to the present invention or the pharmaceutical composition according to the present invention, one or more subsequent administrations may follow, for example a single dose every 2 or 4 weeks for 1, 2, 3, 4, 5, 6, 7, 8, 9, 10, 11, 12, 13, 1, 15, 16, 17, 18, 19, 20, or 21 weeks. After the first administration of the antibody according to the present invention, the nucleic acid according to the present invention, the vector according to the present invention, the cell according to the present invention or the pharmaceutical composition according to the present invention, one or more subsequent administrations may follow, for example a single dose every two or four months for 1, 2, 3, 4, 5, 6, 7, 8, 9, 10, 11, 12, 13, 1, 15, 16, 17, 18, 19, 20, or 21 months. After the first administration of the antibody according to the present invention, the nucleic acid according to the present invention, the vector according to the present invention, the cell according to the present invention or the pharmaceutical composition according to the present invention, one or more subsequent administrations may follow, for example a single dose once or twice per year for 1, 2, 3, 4, 5, 6, 7, 8, 9, or 10 years.

In some embodiments, the antibody according to the present invention, the nucleic acid according to the present invention, the vector according to the present invention, the cell according to the present invention or the pharmaceutical composition according to the present invention is administered at a (single) dose of 0.005 to 100 mg/kg bodyweight or 0.0075 to 50 mg/kg bodyweight, such as at a (single) dose of 0.01 to 10 mg/kg bodyweight or at a (single) dose of 0.05 to 5 mg/kg bodyweight. For example, the antibody according to the present invention, the nucleic acid according to the present invention, the vector according to the present invention, the cell according to the present invention or the pharmaceutical composition according to the present invention is administered at a (single) dose of 0.1 to 1 mg/kg bodyweight.

The antibody according to the present invention, the nucleic acid according to the present invention, the vector according to the present invention, the cell according to the present invention or the pharmaceutical composition according to the present invention may be administered by any number of routes such as oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes.

In some embodiments, the antibody according to the present invention, the nucleic acid according to the present invention, the vector according to the present invention, the cell according to the present invention or the pharmaceutical composition according to the present invention is administered prophylactically, i.e. before diagnosis of influenza A infection.

In some embodiments, the antibody of the invention is administered at a dose which does not exceed half of the dose required for prophylaxis or treatment of influenza A infection with a comparative antibody, which differs from said antibody only in that it does not contain the mutations M428L and N434S in the constant region of the heavy chain. For example, the dose of the antibody of the invention does not exceed one third, one fourth, one fifth, one sixth, one seventh, one eighth or one ninth of the dose required for prophylaxis or treatment of influenza A infection with said comparative antibody. In some embodiments, the antibody of the invention is administered at a dose which does not exceed one tenth of the dose required for prophylaxis or treatment of influenza A infection with a comparative antibody, which differs from said antibody only in that it does not contain the mutations M428L and N434S in the constant region of the heavy chain. Example 5 of the present specification shows that the antibody of the invention comprising the mutations M428L and N434S in the constant region of the heavy chain is effective at much lower doses as compared to a comparative antibody, which differs from the inventive antibody only in that it does not contain the mutations M428L and N434S in the constant region of the heavy chain. Example 5 also shows that the increased efficacy of the antibody of the invention was independent of the circulating antibody levels.

Accordingly, the antibody of the invention may be administered to subjects at immediate risk of influenza A infection. An immediate risk of influenza A infection typically occurs during an influenza A epidemic. Influenza A viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza A epidemic is high during November, December, January, February and March, while in the southern hemisphere the risk of an influenza A epidemic is high during May, June, July, August and September.

Combination Therapy

The administration of the antibody according to the present invention, the nucleic acid according to the present invention, the vector according to the present invention, the cell according to the present invention or the pharmaceutical composition according to the present invention in the methods and uses according to the invention can be carried out alone or in combination with a co-agent (also referred to as “additional active component” herein), which may be useful for preventing and/or treating influenza infection.

The invention encompasses the administration of the antibody according to the present invention, the nucleic acid according to the present invention, the vector according to the present invention, the cell according to the present invention or the pharmaceutical composition according to the present invention, wherein it is administered to a subject prior to, simultaneously with or after a co-agent or another therapeutic regimen useful for treating and/or preventing influenza. Said antibody, nucleic acid, vector, cell or pharmaceutical composition, that is administered in combination with said co-agent can be administered in the same or different composition(s) and by the same or different route(s) of administration. As used herein, expressions like “combination therapy”, “combined administration”, “administered in combination” and the like are intended to refer to a combined action of the drugs (which are to be administered “in combination”). To this end, the combined drugs are usually present at a site of action at the same time and/or at an overlapping time window. It may also be possible that the effects triggered by one of the drugs are still ongoing (even if the drug itself may not be present anymore) while the other drug is administered, such that effects of both drugs can interact. However, a drug which was administered long before another drug (e.g., more than one, two, three or more months or a year), such that it is not present anymore (or its effects are not ongoing) when the other drug is administered, is typically not considered to be administered “in combination”. For example, influenza medications administered in distinct influenza seasons are usually not administered “in combination”.

Said other therapeutic regimens or co-agents may be, for example, an antiviral. An antiviral (or “antiviral agent” or “antiviral drug”) refers to a class of medication used specifically for treating viral infections. Like antibiotics for bacteria, antivirals may be broad spectrum antivirals useful against various viruses or specific antivirals that are used for specific viruses. Unlike most antibiotics, antiviral drugs do usually not destroy their target pathogen; instead they typically inhibit their development.

Thus, in another aspect of the present invention the antibody, or an antigen binding fragment thereof, according to the present invention, the nucleic acid according to the present invention, the vector according to the present invention, the cell according to the present invention or the pharmaceutical composition according to the present invention is administered in combination with (prior to, simultaneously or after) an antiviral for the (medical) uses as described herein.

In general, an antiviral may be a broad spectrum antiviral (which is useful against influenza viruses and other viruses) or an influenza virus-specific antiviral. In some embodiments, the antiviral is not an antibody. For example, the antiviral may be a small molecule drug. Examples of small molecule antivirals useful in prophylaxis and/or treatment of influenza are described in Wu X, Wu X, Sun Q, et al. Progress of small molecular inhibitors in the development of anti-influenza virus agents. Theranostics. 2017; 7(4):826-845. As described in Wu et al., 2017, the skilled artisan is familiar with various antivirals useful in prophylaxis and/or treatment of influenza. Further antivirals useful in influenza are described in Davidson S. Treating Influenza Infection, From Now and Into the Future. Front Immunol. 2018; 9:1946; and in: Koszalka P, Tilmanis D, Hurt A C. Influenza antivirals currently in late-phase clinical trial. Influenza Other Respir Viruses. 2017; 11(3):240-246.

Antivirals useful in prophylaxis and/or treatment of influenza include (i) agents targeting functional proteins of the influenza virus itself and (ii) agents targeting host cells, e.g. the epithelium.

Host cell targeting agents include the thiazolide class of broad-spectrum antivirals, sialidase fusion proteins, type 11 interferons, Bcl-2 (B cell lymphoma 2) inhibitors, protease inhibitors, V-ATPase inhibitors and antioxidants. Examples of the thiazolide class of broad-spectrum antivirals include nitazoxanide (NTZ), which is rapidly deacetylated in the blood to the active metabolic form tizoxanide (TIZ), and second generation thiazolide compounds, which are structurally related to NTZ, such as RM5061. Fludase (DAS181) is an example for sialidase fusion proteins. Type 11 IFNs include, for example, IFNλ. Non-limiting examples of Bcl-2 inhibitors include ABT-737, ABT-263, ABT-199, WEHI-539 and A-1331852 (Davidson S. Treating Influenza Infection, From Now and Into the Future. Front Immunol. 2018; 9:1946). Examples of protease inhibitors include nafamostat, Leupeptin, epsilon-aminocapronic acid, Camostat and Aprotinin. V-ATPase inhibitors include NorakinR, ParkopanR, AntiparkinR and AkinetonR. An example of an antioxidant is alpha-tocopherol.

In some embodiments, the antiviral is an agent targeting a functional protein of the influenza virus itself. For example, the antiviral may target a functional protein of the influenza virus, which is not hemagglutinin. In general, antivirals targeting a functional protein of the influenza virus include entry inhibitors, hemagglutinin inhibitors, neuraminidase inhibitors, influenza polymerase inhibitors (RNA-dependent RNA polymerase (RdRp) inhibitors), nucleocapsid protein inhibitors, M2 ion channel inhibitors and arbidol hydrochloride. Non-limiting examples of entry inhibitors include triterpenoids derivatives, such as glycyrrhizic acid (glycyrrhizin) and glycyrrhetinic acid; saponins; uralsaponins M-Y (such as uralsaponins M); dextran sulphate (DS); silymarin; curcumin; and lysosomotropic agents, such as Concanamycin A, Bafilomycin A1, and Chloroquine. Non-limiting examples of hemagglutinin inhibitors include BMY-27709; stachyflin; natural products, such as Gossypol, Rutin, Quercetin, Xylopine, and Theaflavins; trivalent glycopeptide mimetics, such as compound 1 described in Wu X, Wu X, Sun Q, et al. Progress of small molecular inhibitors in the development of anti-influenza virus agents. Theranostics. 2017; 7(4):826-845; podocarpic acid derivatives, such as compound 2 described in Wu X, Wu X, Sun Q, et al. Progress of small molecular inhibitors in the development of anti-influenza virus agents. Theranostics. 2017; 7(4):826-845; natural product pentacyclic triterpenoids, such as compound 3 described in Wu X, Wu X, Sun Q, et al. Progress of small molecular inhibitors in the development of anti-influenza virus agents. Theranostics. 2017; 7(4):826-845; and prenylated indole diketopiperazine alkaloids, such as Neoechinulin B. Non-limiting examples of nucelocapsid protein inhibitors include nucleozin, Cycloheximide, Naproxen and Ingavirin. Non-limiting examples of M2 ion channel inhibitors include the approved M2 inhibitors Amantadine and Rimantadine and derivatives thereof; as well as non-adamantane derivatives, such as Spermine, Spermidine, Spiropiperidine and pinanamine derivatives.

In some embodiments, the antiviral is selected from neuraminidase (NA) inhibitors and influenza polymerase inhibitors (RNA-dependent RNA polymerase (RdRp) inhibitors). Non-limiting examples of neuraminidase (NA) inhibitors include zanamivir; oseltamivir; peramivir; laninamivir; derivatives thereof such as compounds 4-10 described in Wu X, Wu X, Sun Q et al. Progress of small molecular inhibitors in the development of anti-influenza virus agents. Theranostics. 2017; 7(4):826-845, and dimeric zanamivir conjugates (e.g., as described in Wu X, Wu X, Sun Q et al. Progress of small molecular inhibitors in the development of anti-influenza virus agents. Theranostics. 2017; 7(4):826-845); benzoic acid derivatives (e.g., as described in Wu X, Wu X, Sun Q, et al. Progress of small molecular inhibitors in the development of anti-influenza virus agents. Theranostics. 2017; 7(4):826-845; such as compounds 11-14); pyrrolidine derivatives (e.g., as described in Wu X, Wu X, Sun Q, et al. Progress of small molecular inhibitors in the development of anti-influenza virus agents. Theranostics. 2017; 7(4):826-845; such as compounds 15-18); ginkgetin-sialic acid conjugates; flavanones and flavonoids isoscutellarein and its derivatives (e.g., as described in Wu X, Wu X, Sun Q, et al. Progress of small molecular inhibitors in the development of anti-influenza virus agents. Theranostics. 2017; 7(4):826-845); AV5080; and N-substituted oseltamivir analogues (e.g., as described in Wu X, Wu X, Sun Q et al. Progress of small molecular inhibitors in the development of anti-influenza virus agents. Theranostics. 2017; 7(4):826-845). Non-limiting examples of influenza polymerase inhibitors (RNA-dependent RNA polymerase (RdRp)) inhibitors include RdRp disrupting compounds, such as those described in Wu X, Wu X, Sun Q, et al. Progress of small molecular inhibitors in the development of anti-influenza virus agents. Theranostics. 2017; 7(4):826-845; PB2 cap-binding inhibitors, such as JNJ63623872 (VX-787); cap-dependent endonuclease inhibitors, such as baloxavir marboxil (S-033188); PA endonuclease inhibitors, such as AL-794, EGCG and its aliphatic analogues, N-hydroxamic acids and N-hydroxyimides, flutimide and its aromatic analogues, tetramic acid derivatives, L-742,001, ANA-0, polyphenolic catechins, phenethyl-phenylphthalimide analogues, macrocyclic bisbibenzyls, pyrimidinoles, fullerenes, hydroxyquinolinones, hydroxypyridinones, hydroxypyridazinones, trihydroxy-phenyl-bearing compounds, 2-hydroxy-benzamides, hydroxy-pyrimidinones, 3-diketo acid and its bioisosteric compounds, thiosemicarbazones, bisdihydroxyindole-carboxamides, and pyrido-piperazinediones (Endo-1); and nucleoside and nucleobase analogue inhibitors, such as ribavirin, favipiravir (T-705), 2′-Deoxy-2′-fluoroguanosine (2′-FdG), 2′-substituted carba-nucleoside analogues, 6-methyl-7-substituted-7-deaza purine nucleoside analogues, and 2′-deoxy-2′-fluorocytidine (2′-FdC). For example, the antiviral may be zanamivir, oseltamivir or baloxavir.

Thus, the pharmaceutical composition according to the present invention may comprise one or more of the additional active components. The antibody according to the present invention can be present in the same pharmaceutical composition as the additional active component (co-agent). Alternatively, the antibody according to the present invention and the additional active component (co-agent) are comprised in distinct pharmaceutical compositions (e.g., not in the same composition). Accordingly, if more than one additional active component (co-agent) is envisaged, each additional active component (co-agent) and the antibody, or the antigen binding fragment, according to the present invention may be comprised by a different pharmaceutical composition. Such different pharmaceutical compositions may be administered either combined/simultaneously or at separate times and/or by separate routes of administration.

The antibody according to the present invention and the additional active component (co-agent) may provide an additive or a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Accordingly, the present invention also provides a combination of (i) the antibody of the invention as described herein, and (ii) an antiviral agent as described above.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 2 the plasma concentration of human antibodies FluAB_MLNS (open squares) and FluAB_wt (comparative antibody; filled circles) in macaque plasma samples assessed via ELISA until day 56.

FIG. 2 shows for Example 3 plasma concentrations of FluAB_MLNS (animals C90142, C90190) measured using an anti-CH2 antibody ELISA to quantify total human mAb or HA antigen-binding ELISA to determine functionality of the mAbs. Graphs show linear regression between total human mAb quantification and HA binding for individual animals at selected time points (days 1, 21, 56, 86, and 113).

FIG. 3 shows for Example 4 (A) the concentrations of human antibodies FluAB_MLNS and FluAB_wt in nasal swabs as measured using ELISA and normalized to urea content; and (B) Biodistribution of human antibodies FluAB_MLNS and FluAB_wt, expressed as % urea-normalized concentration in nasal swabs over plasma concentrations. Individual animal IDs and inoculated human antibody variant (FluAB_MLNS or FluAB_wt) are indicated below.

FIG. 4 shows for Example 5 the cumulative bodyweight change over time in Tg32 mice treated with either FluAB_wt (panels B, D, circles), FluAB_MLNS (panels C, E, squares) at 1 mg/kg (panels B, C, grey symbols) and 0.3 mg/kg (panels D, E, light gray symbols) or left untreated (panel A, triangles); all mice infected intranasally with PR8 virus. Individual animals are shown; The thick black line represents the average trend of BW±SD. The number of individuals per group is indicated. * p<0.05, ** p<0.01, *** p<0.001 vs control alone (A), ° p<0.05, ° ° p<0.01, all vs the relative timepoints of MEDI8852, 2-way ANOVA with Bonferroni's multiple test correction.

FIG. 5 shows for Example 5 the % of survival comparison between 1 mg/kg dose (left panel) and 0.3 mg/kg dose (right panel) in infected Tg32 male mice treated with nothing (dashed line), FluAB_wt, or FluAB_MLNS. ** p<0.01 vs untreated mice (CTR) and FluAB_MLNS 0.3 mg/kg; ° ° ° p<0.001 vs FluAB_wt, log-rank analysis, Mantel-Cox method.

FIG. 6 shows for Example 5 the circulating levels of the injected antibodies. The individual levels (μg/ml) of circulating FluAB_wt (circles) and FluAB_MLNS (squares) measured in the serum of mice, immediately before (Day 0) and 6 days after infection are shown. Bars represent the mean±SD.

FIG. 7 shows for Example 6 the plate scheme used in the in vitro neutralization assay.

FIG. 8 shows for Example 6 the neutralization activity of FluAB_MLNS and Oseltamivir alone on H1N1 (A, C) and H3N2 (B, D) virus infection.

FIG. 9 shows for Example 6 the combined neutralization activity of FluAB_MLNS and Oseltamivir on H1 (A) and H3 (B) virus infection. Data show the inhibited fraction by FluAB_MLNS alone and in combination with heteromolar concentrations of Oseltamivir both in H1N1 (A) and H3N2 (B) viral infection of MDCK cells. Data are represented as mean±SD of triplicate values, each replicate obtained in three independent culture plates.

FIG. 10 shows for Example 6 the median effect plots of combined FluAB_MLNS and Oseltamivir. The two compounds were serially diluted at the indicated constant ratios and added to MDCK cells infected with either H1 (A) and H3 (B) viral strains. The values obtained from selected combinations at non-constant ratios (NCR) are also shown.

FIG. 11 shows for Example 6 the combination indexes of FluAB_MLNS and Oseltamivir for H1N1 virus infection. Dots represent the actual experimental points at the indicated constant ratios with the cumulated drug-drug concentration denoted aside. The dotted curves show the predicted combination index across the complete effect range.

FIG. 12 shows for Example 6 the combination indexes of FluAB_MLNS and Oseltamivir for H3N2 virus infection. Dots represent the actual experimental points at the indicated constant ratios with the cumulated drug-drug concentration denoted aside. The dotted curves show the predicted combination index across the complete effect range.

FIG. 13 shows for Example 6 isobolograms of FluAB_MLNS-Oseltamivir combinations for H1N1 virus infection. Dots show the IC₅₀, IC₇₅ and IC₉₀ values on different constant ratio FluAB_MLNS-Oseltamivir combinations. For each experimental point, the cumulated concentration is shown.

FIG. 14 shows for Example 6 isobolograms of FluAB_MLNS-Oseltamivir combinations for H3N2 virus infection. Dots show the IC₅₀, IC₇₅ and IC₉₀ values on different constant ratio FluAB_MLNS-Oseltamivir combinations. For each experimental point, the cumulated concentration is shown.

FIG. 15 shows for Example 6 the neutralization activity of FluAB_MLNS and Zanamivir alone on H1N1 (A, C) and H3N2 (B, D) virus infection.

FIG. 16 shows for Example 6 the combined neutralization activity of FluAB_MLNS and Zanamivir on H1 (A) and H3 (B) virus infection. Data show the inhibited fraction by FluAB_MLNS alone and in combination with heteromolar concentrations of Zanamivir both in H1N1 (A) and H3N2 (B) viral infection of MDCK cells. Data are represented as mean±SD of triplicate values, each replicate obtained in three independent culture plates.

FIG. 17 shows for Example 6 the median effect plots of combined FluAB_MLNS and Zanamivir. The two compounds were serially diluted at the indicated constant ratios and added to MDCK cells infected with either H1 (A) and H3 (B) viral strains. The values obtained from selected combinations at non-constant ratios (NCR) are also shown.

FIG. 18 shows for Example 6 the combination indexes of FluAB_MLNS and Zanamivir for H1N1 virus infection. Dots represent the actual experimental points at the indicated constant ratios with the cumulated drug-drug concentration denoted aside. The dotted curves show the predicted combination index across the complete effect range.

FIG. 19 shows for Example 6 the combination indexes of FluAB_MLNS and Zanamivir for H3N2 virus infection. Dots represent the actual experimental points at the indicated constant ratios with the cumulated drug-drug concentration denoted aside. The dotted curves show the predicted combination index across the complete effect range.

FIG. 20 shows for Example 6 isobolograms of FluAB_MLNS-Zanamivir combinations for H1N1 virus infection. Dots show the IC₅₀, IC₇₅ and IC₉₀ values on different constant ratio FluAB_MLNS-Zanamivir combinations. For each experimental point, the cumulated concentration is shown.

FIG. 21 shows for Example 6 isobolograms of FluAB_MLNS-Zanamivir combinations for H3N2 virus infection. Dots show the IC₅₀, IC₇₅ and IC₉₀ values on different constant ratio FluAB_MLNS-Zanamivir combinations. For each experimental point, the cumulated concentration is shown.

FIG. 22 shows for Example 6 the neutralization activity of FluAB_MLNS and Baloxavir alone on H1N1 (A, C) and H3N2 (B, D) virus infection.

FIG. 23 shows for Example 6 the combined neutralization activity of FluAB_MLNS and Baloxavir on H1 (A) and H3 (B) virus infection. Data show the inhibited fraction by FluAB_MLNS alone and in combination with heteromolar concentrations of Baloxavir both in H1N1 (A) and H3N2 (B) viral infection of MDCK cells. Data are represented as mean±SD of triplicate values, each replicate obtained in three independent culture plates.

FIG. 24 shows for Example 6 the median effect plots of combined FluAB_MLNS and Baloxavir. The two compounds were serially diluted at the indicated constant ratios and added to MDCK cells infected with either H1 (A) and H3 (B) viral strains. The values obtained from selected combinations at non-constant ratios (NCR) are also plotted.

FIG. 25 shows for Example 6 the combination indexes of FluAB_MLNS and Baloxavir. Dots represent the actual experimental points at the indicated constant ratios with the cumulated drug-drug concentration denoted aside. The dotted curves show the predicted combination index across the complete effect range.

FIG. 26 shows for Example 6 isobolograms of FluAB_MLNS-Baloxavir combinations. Dots show the IC₅₀, IC₇₅ and IC₉₀ values on different constant ratio FluAB_MLNS-Baloxavir combinations. For each experimental point, the cumulated concentration is shown.

FIG. 27 shows for Example 7 the binding of human FcRn in solution to immobilized FluAB_MLNS (gray line) or FluAB_wt (black line) as measured by Octet at pH=6.0 (A) or pH=7.4 (B). The time point 0 seconds represents switch from base line buffer to buffer containing human FcRn. Time point 420 seconds (gray dotted vertical line) represents switch to blank buffer at the corresponding pH. Association and dissociation profiles were measured in real time using an Octet RED96 (ForteBio).

FIG. 28 shows for Example 9 the levels of ADA response measured by ELISA to detect mouse anti-drug IgG (A; bars represent the mean±SD of treatment group); and correlation analysis (B) between the levels of circulating human IgG measured 14 days after i.v. injection (X axis) and the signal of the ADA present at the same time point (Y axis). The non-parametric Spearman's correlation coefficient is shown for the significant values.

FIG. 29 shows for Example 10 levels of ADA response after subcutaneous (s.c.) injection of either FluAB_MLNS or FluAB_wt. Data are represented as values of the ADA signal (OD 450 nm) detected in each individual serum obtained three weeks after the s.c. injection (n=5/group), pre-diluted in PBS 1:25 and then further serially diluted 5-fold.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Safety and Tolerability of an Antibody According to the Present Invention in Cynomolgus Macagues

An antibody according to the present invention, which comprises (i) the CDR sequences as set forth in SEQ ID NOs 1-6 and (ii) the two mutations M428L and N434S in the heavy chain constant regions, was designed and produced. More specifically, the antibody comprises (i) the heavy chain variable region (VH) sequence as set forth in SEQ ID NO: 7 and the light chain variable region (VL) sequence as set forth in SEQ ID NO: 8; and (ii) the two mutations M428L and N434S in the heavy chain constant regions. Even more specifically, the antibody comprises a heavy chain having an amino acid sequence as set forth in SEQ ID NO: 9 and a light chain having an amino acid sequence as set forth in SEQ ID NO: 10. This antibody is referred to herein as “FluAB_MLNS”.

For comparison, antibody “FluAB_wt” was used, which differs from antibody “FluAB_MLNS” only in that it does not contain the two mutations M428L and N434S in the heavy chain constant regions. Accordingly, comparative antibody “FluAB_wt” comprises a heavy chain having an amino acid sequence as set forth in SEQ ID NO: 11 and a light chain having an amino acid sequence as set forth in SEQ ID NO: 10.

A single intravenous infusion of 5 mg/kg of either FluAB_MLNS or FluAB_wt in a 2.5 ml/kg volume was given in a 60-minutes intravenous infusion to three female cynomolgus macaques (Macaca fascicularis) per test group. Blood or urine for clinical chemistry and hematological analyses were collected pre-dose and on days 7 and 21 post-dose.

Following dosing of either FluAB_MLNS or FluAB_wt at 5 mg/kg in a 60-minutes intravenous infusion, the female cynomolgus macaques were closely monitored for health and weight and regularly sampled for blood and urine. No adverse events—other than bruising 24 h and erythroderma 3 days post-dose at the inoculation site in some of the animals—were observed following intravenous inoculation of the antibodies. All animals were generally healthy, showed normal food consumption, and had overall positive weight gain throughout the study. Clinical chemistry, hematology, and urinalysis parameters were normal at 7- or 21-days post dosing, compared to pre-dosing samples.

In summary, a single intravenous infusion of either FluAB_MLNS or FluAB_wt into cynomolgus macaques did not induce adverse events and was generally well tolerated.

### Example 2: Determination of Plasma Concentration and Pharmacokinetics

These experiments aimed to determine the concentration, establish half live, and compare the pharmacokinetics of the antibody according to the present invention FluAB_MLNS in comparison to comparative antibody FluAB_wt in the plasma following a single intravenous injection.

Before dosing, the animals were tested to be negative for influenza-specific antibodies using dot immunobinding assay. Seropositive animals were excluded from the study as pre-existing immunity may interfere with this test. In addition, animals developing anti-drug antibody (ADA) response were excluded.

A single intravenous infusion of 5 mg/kg of either FluAB_MLNS or FluAB_wt in a 2.5 ml/kg volume was given in a 60-minutes intravenous infusion to three female macaques per test group. Blood was collected in tubes containing K₂EDTA pre-dose and processed to plasma for pharmacokinetic testing after approximately 1, 6, 24, 96, 168, 504, 840, and 1344 hours (h) post-dose.

Plasma concentration of the antibodies was determined in vitro using an ELISA assay. Briefly, IAV-HA antigen (Influenza A virus H1N1 A/California/07/2009 Hemagglutinin Protein Antigen (with His Tag); Sino Biologicals) was diluted to 2 μg/ml in PBS and 25 μl were added to the wells of a 96-well flat bottom %-area ELISA plate for coating over night at 4° C. After coating, the plates were washed twice with 0.5×PBS supplemented with 0.05% Tween20 (wash solution) using an automated ELISA washer. Then, plates were blocked with 100 μl/well of PBS supplemented with 1% BSA (blocking solution) for 1 h at room temperature (RT) and then washed twice. Plasma samples were centrifuged at 10′000 g for 10 min at 4° C. and then diluted (1:10 and then 1:30) for a final 1:300 dilution in blocking solution in 96-well cell culture plates. The minimum dilution (1:300) of the macaque plasma used for quantification was tested and set to ensure that the matrix effect was negligible. Samples were then diluted 1:2 stepwise in triplicates for a total of 12 dilutions. Standards for each antibody to be tested were prepared similarly via diluting the antibodies 1:300 to 1 μg/ml in a pool of pre-inoculation plasma from all test animals, mimicking the matrix of the test samples. Standards were then diluted 1:3 stepwise in blocking solution in triplicates for a total of 12 dilutions. Twenty-five μl of the prepared samples or standards were added to hemagglutinin (HA)-coated wells and incubated for 1 h at RT. After four washes, 25 μl of goat anti human-IgG HRP conjugate (AffiniPure F(ab′)₂ Fragment, Fcγ Fragment-Specific; Jackson ImmunoResearch) diluted in blocking solution 1:5′000 (final concentration 0.16 μg/ml) were added per well for detection and incubated at RT for 1 h. After four washes, plates were developed by adding 40 μl per well of SureBlue TMB Substrate (Bioconcept). After ˜7-20 min incubation at RT, when the color reaction reached a plateau (max OD˜3.8), 40 μl of 1% HCl were added per well to stop the reaction and absorbance was measured at 450 nm using a spectrophotometer.

To determine the concentration of the antibodies in cynomolgus plasma, OD values from ELISA data were plotted vs. concentration in the Gen5 software (BioTek). A non-linear curve fit was applied using a variable slope model, four parameters and the equation: Y=(A−D)/(1+(X/C)\hat{ }B)+D).

The OD values of the sample dilutions that fell within the predictable assay range of the standard curve as determined in setup experiment by quality control samples in the upper, medium or lower range of the curve were interpolated to quantify the samples. Plasma concentration of the antibodies were then determined considering the final dilution of the sample. If more than one value of the sample dilutions fell within the linear range of the standard curve, an average of these values was used. Pharmacokinetics (PK) data were analyzed by using WINNONLIN NONCOMPARTMENTAL ANALYSIS PROGRAM (8.1.0.3530 Core Version, Phoenix software, Certara) with the following settings: Model: Plasma Data, Constant Infusion Administration; Number of non-missing observations: 8; Steady state interval Tau: 1.00; Dose time: 0.00; Dose amount: 5.00 mg/kg; Length of Infusion: 0.04 days; Calculation method: Linear Trapezoidal with Linear Interpolation; Weighting for lambda_z calculations: Uniform weighting; Lambda_z method: Find best fit for lambda_z, Log regression. Graphing and statistical analyses (linear regression or outlier analysis) were performed using Prism 7.0 software (GraphPad, La Jolla, Calif., USA). Outlier analysis was performed using the ROUT method (Q=1%), with the potential to find any number of outliers in either direction.

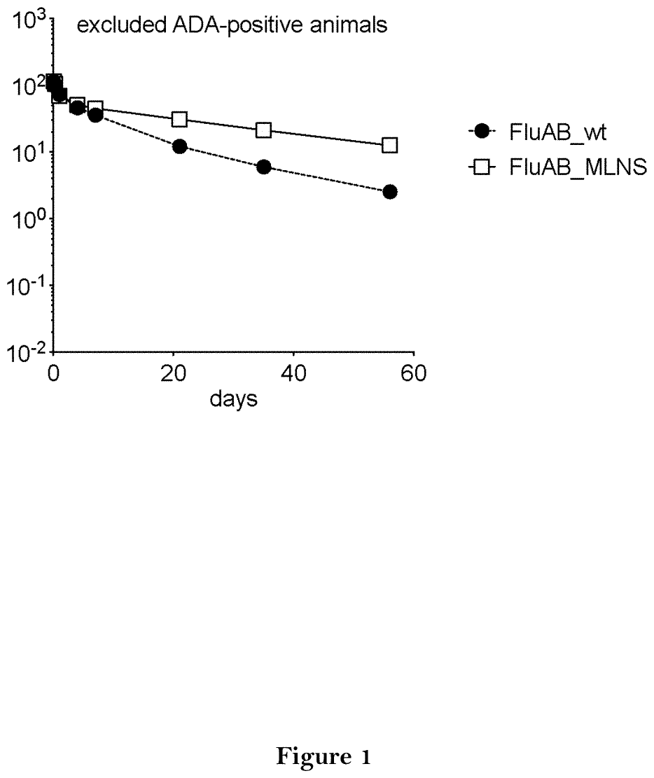

Results are shown in FIG. 1. Analysis of cynomolgus plasma samples drawn up to 56 days post-inoculation demonstrated that the antibody according to the present invention FluAB_MLNS had an extended in-vivo half-live compared to comparative antibody FluAB_wt (FIG. 1). Using noncompartmental analysis with WinNonLin, the T_{1/2} was estimated as 19.5 days for the antibody according to the present invention FluAB_MLNS, while T_{1/2} was estimated as 11.6 days for the comparative antibody FluAB_wt. The lower limit of quantification was 300 ng/ml.

In summary, the antibody according to the present invention FluAB_MLNS had an extended in-vivo half-live compared to comparative antibody FluAB_wt at least up to day 56 post-inoculation.

### Example 3: Long-Term Stability In Vivo

To test in-vivo stability and functionality of the antigen binding of the antibody according to the present invention FluAB_MLNS over time, the pharmacokinetics measurement (as described in Example 2) of the group receiving the antibody according to the present invention FluAB_MLNS was extended to days 86 and 113 post-inoculation. On days 1, 21, 56, 86, 113 post-inoculation, functional FluAB_MLNS was quantified using the hemagglutinin (HA) binding ELISA as described in Example 2.

Further, total human antibodies in macaque plasma was quantified using a specific anti-CH2 ELISA, using a capture mAb that specifically binds the CH2 region of human but not of monkey Abs. To measure total human IgG and thus quantify total inoculated human antibodies in cynomolgus plasma, an ELISA capturing with mouse anti-CH2 domain-specific to human IgG (clone R10Z8E9; Thermo Scientific) was used. It was verified that this mAb does not cross-react with monkey IgG. For coating of 96-well flat bottom %-area ELISA plates, mouse anti-human IgG CH2 was added in PBS at 0.5 μg/ml and incubated over night at 4° C. Then, plates were washed and 100 μl/well blocking solution with 5% BSA was added for 1 h at RT. Standards of the antibody according to the present invention FluAB_MLNS were prepared via diluting the FluAB_MLNS to 1 ng/ml in blocking solution. Standards were then diluted 1:1.5 stepwise in blocking solution in duplicates for a total of 12 dilutions. Cynomolgus plasma samples were centrifuged at 10,000 g for 10 min at 4° C. and step-wise diluted to a final 1:1,000, 1:5,000 or 1:15,000 in blocking solution. After washing the plate, 25 μl of samples or standard were added to the ELISA plate and incubated for 1 h at RT. After three washes, 25 μl of goat anti human-IgG HRP (AffiniPure F(ab′)₂ Fragment, Fcγ Fragment-Specific; Jackson ImmunoResearch) at 0.04 μg/ml were added in blocking solution with 1% BSA for detection and incubated at RT for 45 min. After three washes, plates were developed by adding 40 μl per well of SureBlue TMB Substrate (Bioconcept). After 20 min incubation at RT, 40 μl of 1% HCl were added to stop the reaction, and absorbance was measured at 450 nm.

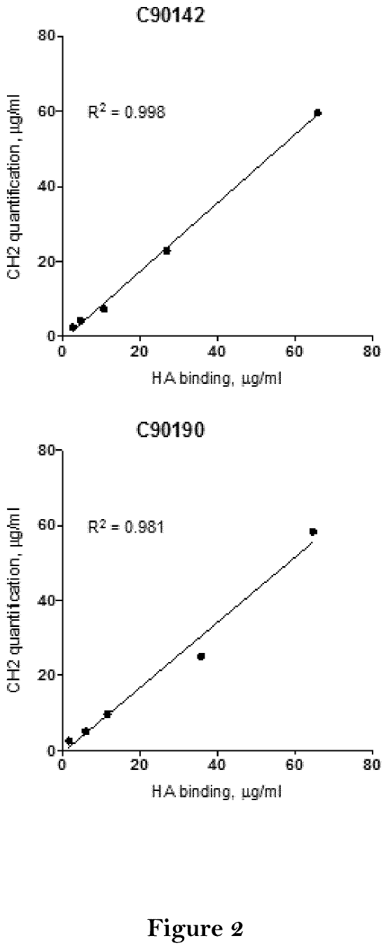

Results are shown in FIG. 2. Both quantifications resulted in similar human antibody concentrations in cynomolgus plasms (FIG. 2). Additional analysis via linear regression demonstrated that the relation between quantification via HA binding and total anti-CH2 quantification followed a linear patter for all selected time points. Consequently, the total amount of FluAB_MLNS present in plasma was functional in binding to the hemagglutinin (HA) stem region of influenza A virus (IAV), also after 86 and 113 days in vivo.

In summary, the antibody according to the present invention FluAB_MLNS demonstrated functional antigen binding and thus good long-term stability in vivo up to day 113 post-inoculation during study extension.

### Example 4: Antibody Concentration in Nasal Swabs and Biodistribution

To determine biodistribution of the antibody according to the present invention FluAB_MLNS and of the comparative antibody FluAB_wt between the nasal mucus relative to plasma, the concentration of the antibody was determined in nasal swabs. To this end, Nasal swabs of the macaques described in Example 2 were collected 24, 504, and 1344 hours after administration of the antibody according to the present invention FluAB_MLNS or of the comparative antibody FluAB_wt. Concentrations of antibodies FluAB_MLNS and FluAB_wt in nasal swabs were determined essentially as described in Example 2 for determination in plasma with the following minor adaptations: (a) ELISA plates were blocked 2 h at RT; (b) Nasal swab samples were diluted starting at 1:2 with 1% BSA in PBS and then serially diluted step-wise 1:2 for a total of 8 dilution points; (c) nasal swab medium (RT MINI Viral Transport Medium; Copan) was used as assay matrix control.

To eliminate differences during the swabbing procedure or in the amount of nasal secretions present in each animal and at different time points (days 1, 21, and 56), results from nasal swabs were normalized to urea content. Urea freely diffuses between blood, being present in similar amounts across these plasma or swab samples (Lim et al., 2017, Antimicrob Agents Chemother 61(8):e00279-17). To this end, Urea Nitrogen (BUN) was measured quantitatively using the “Urea Nitrogen (BUN) Colorimetric Detection Kit” (Invitrogen), following the manufacture's procedure. In brief, samples were diluted 1:3 in PBS and mixed with the kit reagents A and B and incubated at room temperature for 30 minutes. The colored product of the redox reaction was read at 450 nm using a 96-well microplate reader. Quantification was performed via comparing samples to BUN standards, which were provided with the kit and treated equivalently.

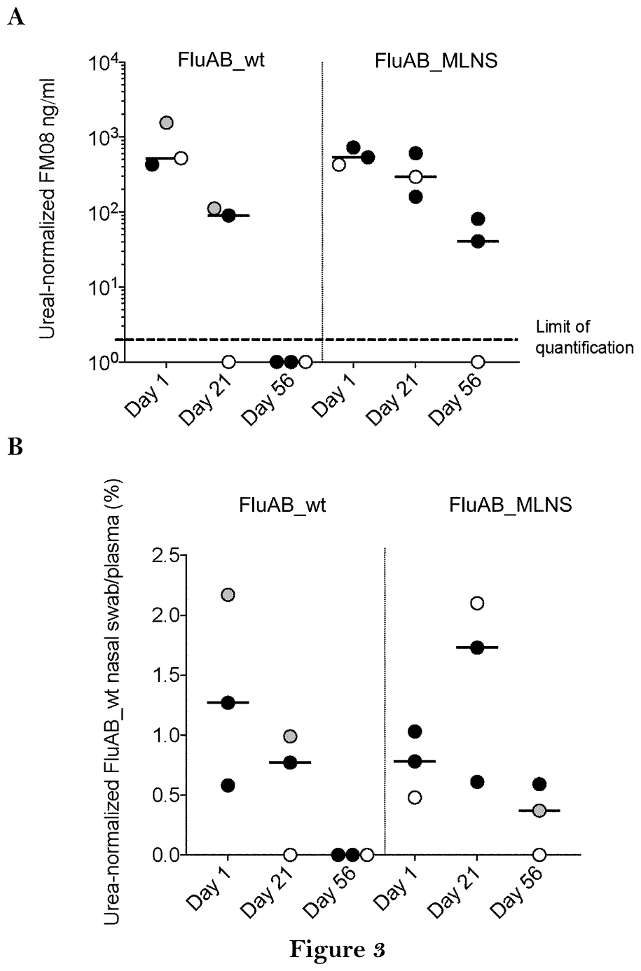

Results are shown in FIG. 3. Amounts of normalized antibodies in nasal swabs decreased over time (FIG. 3A). Determining biodistribution via comparing nasal to plasma concentrations revealed no differences between the antibody according to the present invention FluAB_MLNS and the comparative antibody FluAB_wt (FIG. 3B), suggesting that the MLNS-Fc mutation, while prolonging the half-life of FluAB_MLNS in plasma, did not enhance bio-distribution of the antibody into the nasal mucus.

In summary, nasal swab samples did not reveal any significant differences in biodistribution between the nasal mucus and plasma amongst the three mAb variants.

### Example 5: Prophylactic Activity of Antibody FluAB_MLNS in PR8-Infected Tg32 Mice

Next, the prophylactic activity of the antibody according to the present invention FluAB_MLNS compared to antibody FluAB_wt was determined in a H1N1 murine model of lethal influenza A infection.

To evaluate the prophylactic efficacy, 9- to 14-week-old FcRn−/− hFcRn line 32 Tg mice (C57B6 background) were intravenously (i.v.)-injected (via the tail vein) with 5 ml/kg of a solution containing the antibody according to the present invention FluAB_MLNS or the comparative antibody FluAB_wt at doses ranging from 0.3 to 1 mg/kg. Twenty-four hours after the i.v. injection, mice were bled from the tail vein to determine the serum antibody levels before infection. Bleedings were also repeated on day 6 and 13 post infection (p.i.). Both antibody-injected and untreated mice were anaesthetized (isoflurane, 4% in O₂, 0.3 L/min) and challenged intranasally (i.n.) by slow instillation in both nostrils of 50 μl (25 al/each) of PBS containing 5 mouse lethal dose fifty percent (5 MLD₅₀, equivalent to 1200 TCID₅₀/mouse) of influenza virus A (H1N1, A/Puerto Rico/8/34, as described in Cottey, R., Rowe, C. A., and Bender, B. S. (2001). Influenza virus. Curr Protoc Immunol Chapter 19, Unit 19.11-19.11.32). Each mouse was held upright with its head tilted slightly back for about 1 minute to reduce the likelihood of inoculum dripping from the nares. After the procedure and upon righting reflex occurrence, animals were returned to the cage. The mice were monitored daily for weight loss and disease symptoms until day 14 p.i. and euthanized if they lost more than 20% of their initial body weight (whereby 0% is set on the day of infection) or reached morbidity score of 4. Table 1 details the applied morbidity score:

TABLE 1
Morbidity Score of PR8-infected mice

| Morbidity | |
|---|---|
| Score | Clinical signs |
| 1 | Healthy |
| 2 | Consistently ruffled fur on the neck |
| 3 | Piloerection, possible deeper |
| | breathing, less alert |
| 4 | Labored breathing, tremors |
| | and lethargy |
| 5 | Abnormal gait, reduced mobility, |
| | emaciation, tail-ears cyanosis |
| 6 | Death |

All the animals were eventually sacrificed to collect serum and lungs.

Serum Preparation:

Approximately 0.05 ml of blood were collected into gel-containing tubes and let stay for 30 min at RT. Tubes were spun for 5 min at 5500 rpm (3200×g), serum was transferred to new tubes and stored at −20° C. until use.

Two independent experiments were carried out, according to the following designs:

TABLE 2
Study Design Experiment 1:

| Group | N of animals | IV Treatment | mAb Dose | |
|---|---|---|---|---|
| 1 | 4 | — | — | |
| 2 | 8 | FluAB_wt | 1 | mg/kg |
| 3 | 4 | FluAB_wt | 0.3 | mg/kg |
| 4 | 8 | FluAB_MLNS | 1 | mg/kg |
| 5 | 4 | FluAB_MLNS | 0.3 | mg/kg |

TABLE 3
Study Design Experiment 2:

| Group | N of animals | IV Treatment | mAb Dose |
|---|---|---|---|
| 1 | 9 | — | — |
| 2 | 10 | FluAB_wt | 0.3 mg/kg |
| 3 | 6 | FluAB_MLNS | 0.3 mg/kg |

ELISA Quantification of Circulating mAb:

Sera were assessed for the levels of circulating antibodies on day 0 and 6. Briefly, half-area ELISA plates were coated over night at 4° C. with recombinant hemagglutinin (HA) from H1N1 strain A/California/07/09 (2 [symbol] g/ml, in PBS, 25 μl/well). Following blocking (PBS/1% BSA, 100 μl/well, 1 hr RT) and 2 washes (220 [symbol] /well) with ELISA washing solution (PBST), both dilutions of the sera (initial dilution 1:150 for 1 mg/kg, 1:50 for 0.3 mg/kg) and the antibody standards (FluAB_MLNS and FluAB_wt, 0.1 [symbol] g/ml) were added (25 [symbol] /well) in duplicate and serially diluted (1:2 by 10 points for serum dilutions, 1:3 by 8 points for antibody standards). After 1.5 hr RT incubation, plates were washed 4 times with PBST and further incubated 1.5 hr at RT with the HRP-labeled anti-human secondary antibody (0.16 Mg/ml, 25 DI/well). After 4 washes with PBST, plates were dispensed with substrate solution (25 DI/well), developed for 14 min and blocked with 1% HCl (v/v, 25 DI/well). Plates were finally read at 450 nm with a spectrophotometer for signal quantification. Concentration values were calculated by using a non-linear regression model (variable slope model, four parameters, GraphPad Prism) of log (agonist) versus response.

Data Analysis:

Data were plotted and analyzed using GraphPad Prism software version 8.0 for Macintosh, GraphPad Software, La Jolla Calif. USA, www.graphpad.com. Continuous variables were assessed for statistically significant difference (p<0.05, 95% confidence interval) by using ordinary 2-way ANOVA corrected with Bonferroni multiple comparison test. Survival data were compared by using log-rank analysis with Mantel-Cox method (p<0.05 considered statistically significant). The data from the two independent experiments described above were pooled.

Results:

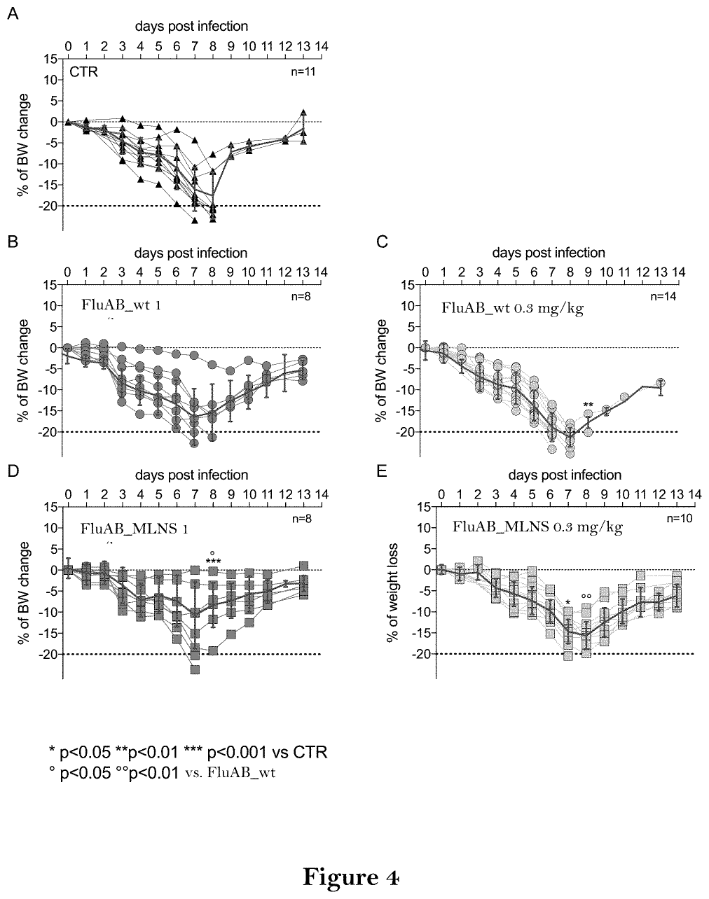

The prophylactic activity was tested upon i.v. administration of FluAB_MLNS and FluAB_MLNS (1 and 0.3 mg/kg) in Tg32 mice one day prior to H1N1 PR8 virus challenge via intranasal infection. Results are shown in FIGS. 4-6.

As depicted in FIG. 4, mice treated with either 1 mg/kg (panel D) or 0.3 mg/kg (panel E) of FluAB_MLNS showed lower body weight loss, in comparison with both untreated (panel A) and FluAB_wt-injected (panels B and C) mice.

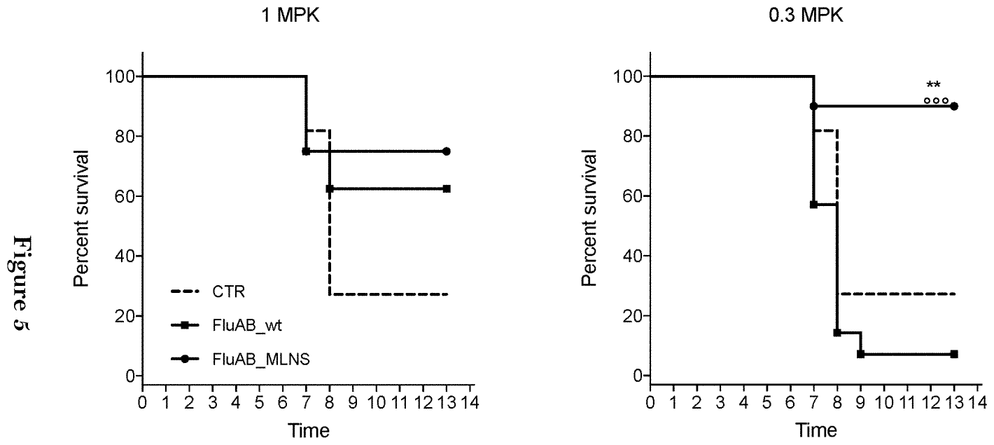

The better protective activity of FluAB_MLNS as compared to FluAB_wt was confirmed in the survival analysis shown in FIG. 5.

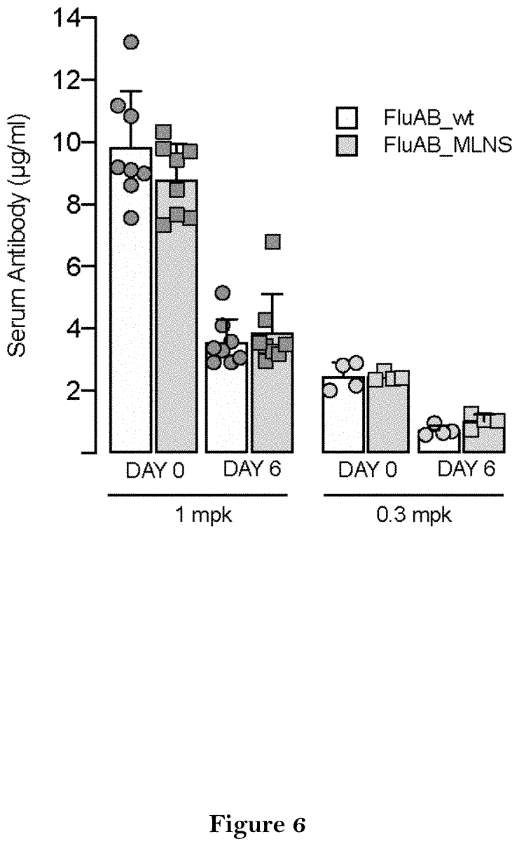

The differences in the efficacy between FluAB_MLNS and FluAB_wt did not correlate with different levels of circulating antibodies in the serum, as measured 1 and 7 days after i.v. administration of the antibodies (FIG. 6). Of note, no detectable levels of circulating antibodies were measured 14 days after injection (not shown).

In summary, FluAB_MLNS demonstrated, in Tg32 mice, a better protective capacity against H1N1 PR8 intranasal virus challenge over the comparative antibody FluAB_wt. The efficacy was independent of the circulating antibody levels. These data suggest that the enhanced interaction of FluAB_MLNS with hFcRn expressed by Tg32 mice also mediates in vivo effects unrelated to the extended antibody half-life, such as increased efficacy regarding the protective activity.

### Example 6: Combination of Antibody FluAB_MLNS with Various Antivirals

Drug combinations offer the clear opportunity to enhance the potency while reducing the probability to select resistances. Moreover, a putative additive or synergic effect may end up to a dose-sparing approach. Influenza medications currently approved by FDA include the neuraminidase inhibitors oseltamivir and zanamivir as well as the recently approved baloxavir marboxil, which belongs to the endonuclease inhibitors class.

To evaluate the combined activity of the antibody of the invention FluAB_MLNS with the antivirals oseltamivir, zanamivir or baloxavir marboxil on both H1N1 and H3N2 representative viral strains, in vitro neutralization was performed to evaluate the resulting inhibitory effect. The analysis of the combined effects was carried out by using the median-effect plot and the calculation of the combination index (CI).

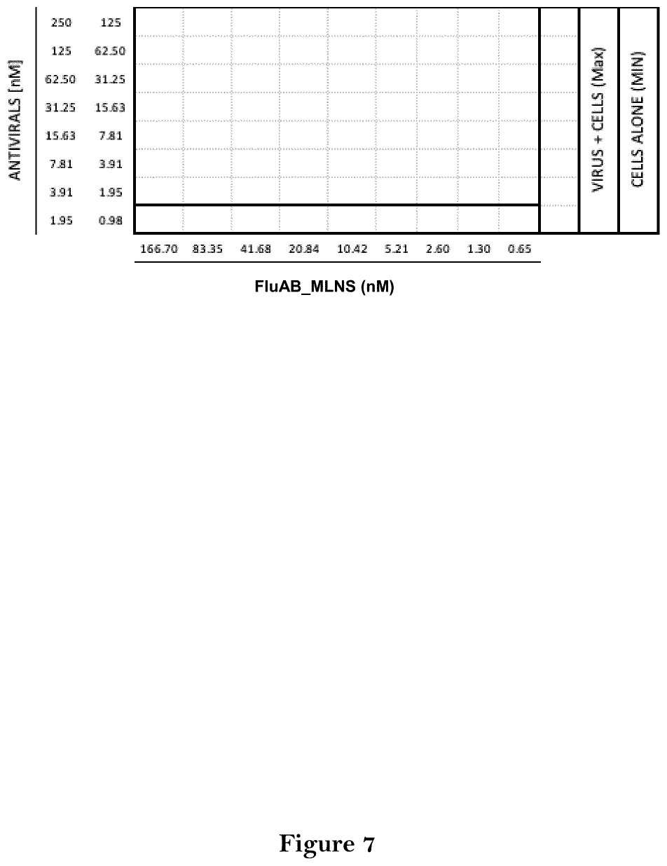

Briefly, MDCK (Madin-Darby canine kidney) cells were seeded at 30,000 cells/well into 96-well plates (flat bottom, black). Cells were cultured at 37° C. 5% CO₂ overnight. Twenty-four hours later, 4× antibody and antiviral (oseltamivir, zanamivir or baloxavir marboxil) dilutions in 60 μl infection medium (MEM (Sigma Aldrich, cat. n. M0644)+Glutamax (Invitrogen, 41090-028)+10 [symbol] g/ml TPCK-treated Trypsin (Worthington Biochemical #LS003750)+10 [symbol] g/ml Kanamycin) were prepared by using crisscross 1:2 serial dilutions of FluAB_MLNS (starting from 166.7 nM final, 9 horizontal points) and different antivirals (oseltamivir, zanamivir or baloxavir marboxil), starting from 125 (250 for zanamivir) nM by 7 vertical points), according to the plate scheme shown in FIG. 7.

For each combination, three independent plates were prepared, in order to have triplicates of each drug-drug combination ratio. The single compound titration (namely, FluAB_MLNS, 9 points and each antiviral, 8 points) was included in each plate. Virus solution was prepared at concentrations of 120× the TCID50 in 60 μl, further diluted either 1:1 in MEM or mixed 1:1 with FluAB_MLNS dilutions and incubated 1 h at 33° C. Cells were washed 2 times using 200 μl/well MEM without supplements, followed by the addition of either 100 μl of virus alone or 100 μl of FluAB_MLNS/virus mix (100×TCID50/well) and incubated 4 hours at 33° C. 5% CO2. After the addition of 100 μl/well of infection medium, cells were further incubated for 72 hours at 33° C. 5% C02. On day 3 after infection, 20 μM MuNANA (4-MUNANA (2_-(4-Methylumbelliferyl)-α-D-N-acetylneuraminic acid sodium salt hydrate (Sigma-Aldrich) #69587) solution was prepared in MuNANA buffer (MES 32.5 mM/CaCl₂ 4 mM, pH 6.5) and 50 μl/well was dispensed into black 96-well plates. Fifty μl of either neutralization or virus-alone titration supernatant were transferred to the plates and incubated 60 min at 37° C. The reaction was then stopped with 100 μl/well 0.2 M glycine/50% EtOH, pH 10.7. Fluorescence was quantified at 460 nm with a fluorimeter (Bio-Tek).

The fraction of virus neutralization was calculated according to the formula:

$$1 - ( \frac{f x - f min}{f max} ) ,$$

wherein fx=sample fluorescence signal (cells+virus+FluAB_MLNS+antiviral); fmin=minimal fluorescence signal (cells alone, no virus); fmax=maximal fluorescence signal (cells+virus only).

The neutralized fraction data were used to compute the quantitative analysis of dose-effect relationships for drug-drug combinations according to the Chou and Talalay method (Chou T C, Talalay P: Quantitative analysis of dose-effect relationships: the combined effects of multiple drugs or enzyme inhibitors. Adv. Enzyme Regul. 1984, 22:27-55). The combination Index, the fraction affected (Fa), and isobolograms were obtained by using the CompuSyn software (ComboSyn Inc., Paramus, N.J., USA) (Chou T-C: Theoretical basis, experimental design, and computerized simulation of synergism and antagonism in drug combination studies. Pharmacological Reviews 2006, 58:621-681).

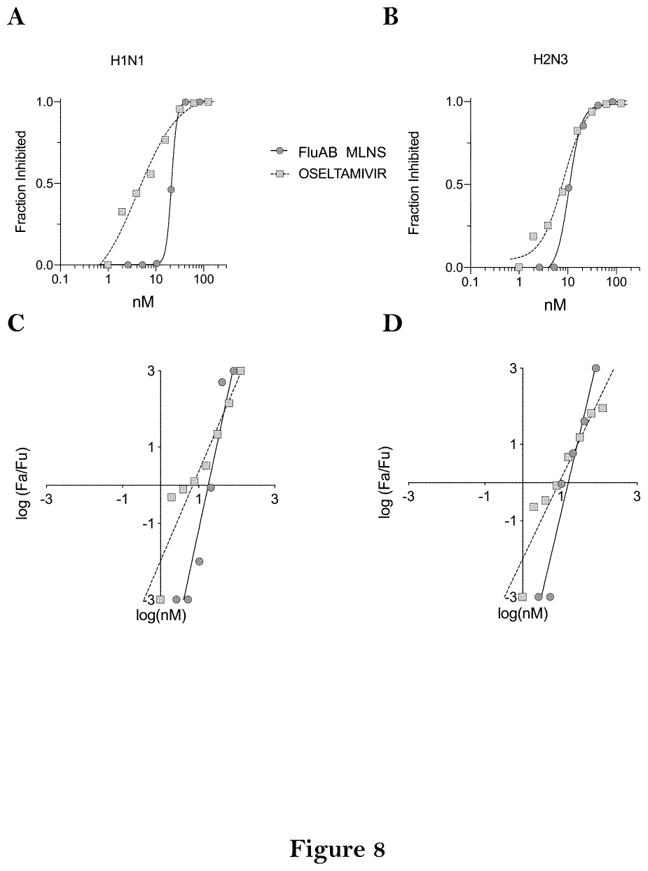

Results are shown in FIGS. 8-26 and described below.

Combination of FluAB_MLNS and Oseltamivir

The relative efficacy of FluAB_MLNS and oseltamivir to neutralize influenza A viruses was compared in vitro on two viral serotype representatives for both H3N2 and H1N1 strains. As shown in FIG. 8, both compounds, tested separately, were dose-dependently capable to fully inhibit cell infection, when independently exposed together with H3N3 and H1N1 virus (FIG. 8A,B). The IC₅₀ values, as calculated from the median-effect plot (FIG. 8C,D) after data log linearization (as described in (Chou T C, Talalay P: Quantitative analysis of dose-effect relationships: the combined effects of multiple drugs or enzyme inhibitors. Adv. Enzyme Regul. 1984, 22:27-55) are indeed in the nanomolar range for both FluAB_MLNS (17.9 and 15.6 nM for H3 and H1 strains respectively) and oseltamivir (7 and 9.1 nM for H3 and H1 strains respectively). Overall, no substantial differences were measured in terms of inhibitory response by FluAB_MLNS between H3 and H1 virus infection, while H1N1 virus resulted marginally more sensitive to the inhibitory effect of oseltamivir.

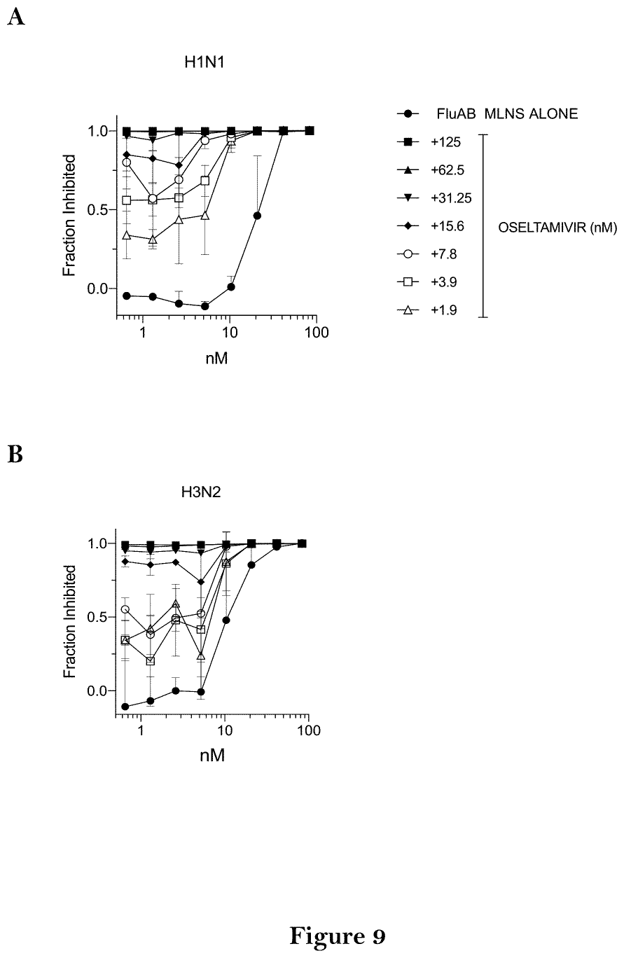

To test the effect of a combination of FluAB_MLNS and oseltamivir in neutralizing the infection of MDCK cells with H3 and H1 virus, both compounds were serially diluted at different ratios as described above, and assessed for the enzymatic activity of neuraminidase (NA; as a read out of the viral content in the culture) in the presence of the different drug concentrations and compared to the single drug effects. The neutralization effect measured with FluAB_MLNS is greatly enhanced by the concomitant presence of heteromolar concentrations of the second compound, thus suggesting a synergistic effect rather than an addictive one, both on H3 and H1 virus infection (FIG. 9). A slightly different susceptibility of H1 and H3 viruses to the inhibitory action of oseltamivir was detected.

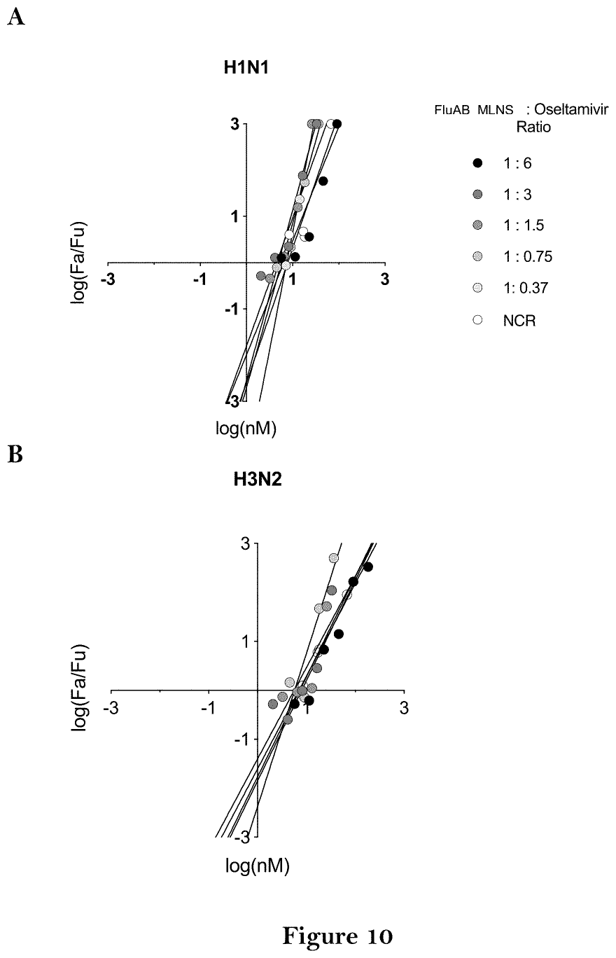

To precisely quantify the putative synergistic effects of the various drug combination ratios, the neutralization data were further transformed according to the median-effect principle and analyzed with the CompuSyn software as described above. The effects of several different FluAB_MLNS-oseltamivir combination constant ratios were plotted in the median-effect plot as shown in FIG. 10.

The CompuSyn software applies the logarithmic transformation of the median-effect equation to the experimental data and calculates both the potency (IC₅₀) and the so-called combination index (CI) of the various drug combinations. The CI is a Chou-Talalay (median-effect) equation-derived parameter that considers the physico-chemical properties of the mass-action law and results from the sum of the two ratios between the portion of the dose of drug 1 combined with drug 2 to achieve a certain effect divided by dose of the single drug 1 and 2 to obtain the same effect. According to this mathematical algorithm, a CI=1 indicates an addictive effect, CI<1 indicates synergism and CI>1 indicates antagonism.

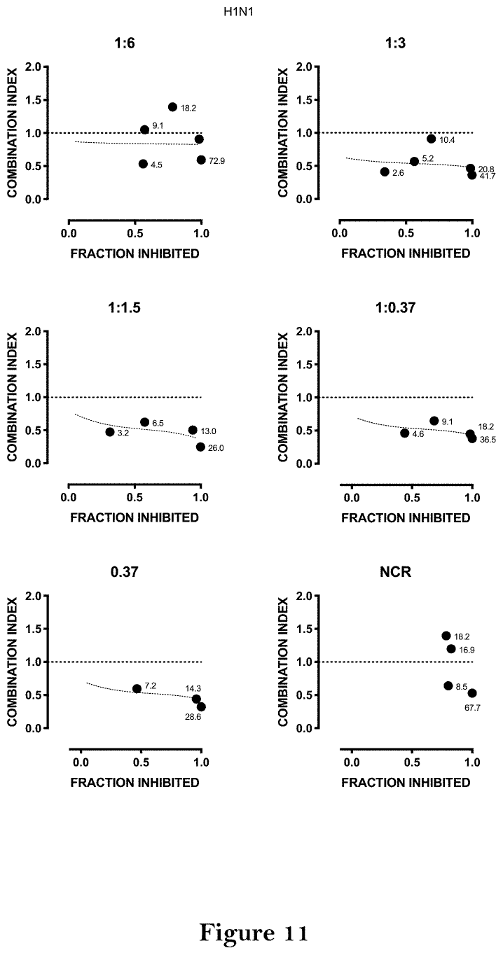

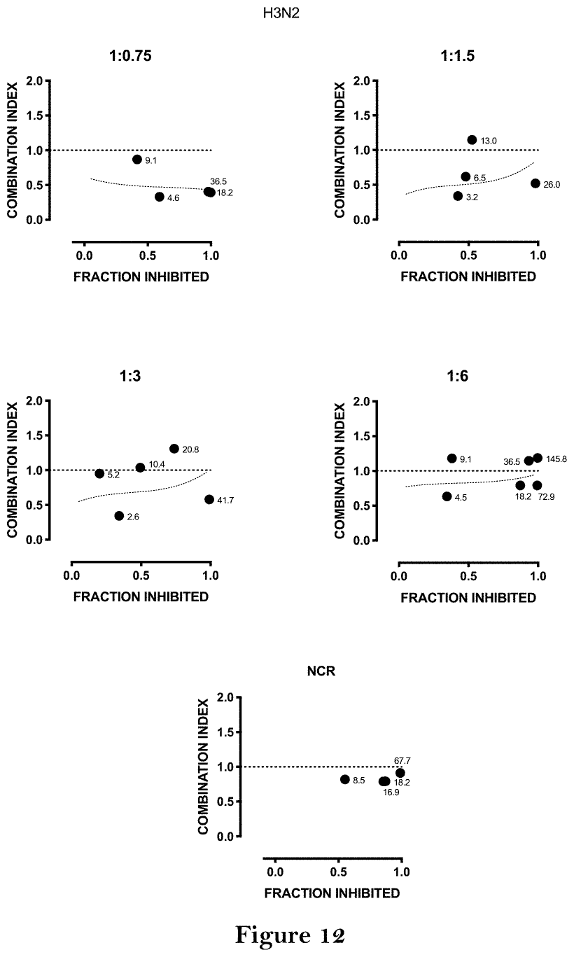

As shown in FIGS. 11 and 12, for all the combination ratios tested and both for H1 (FIG. 11) and H3 virus (FIG. 12), the predicted CI values across the inhibited fraction range described a curve well below 1 for all drug combination ratios and the actual experimental points of the different combined concentrations also ranged below 1 for nearly all combinations. Altogether, the data indicate a straightforward synergistic effect of FluAB_MLNS and oseltamivir when combined.

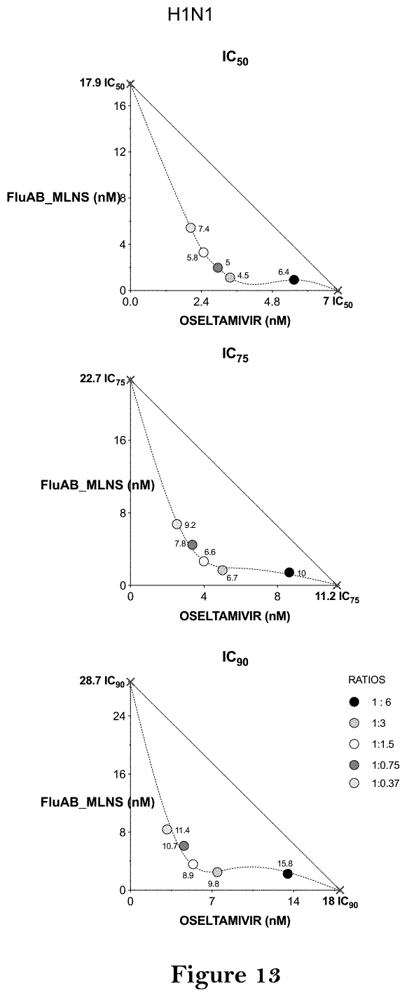

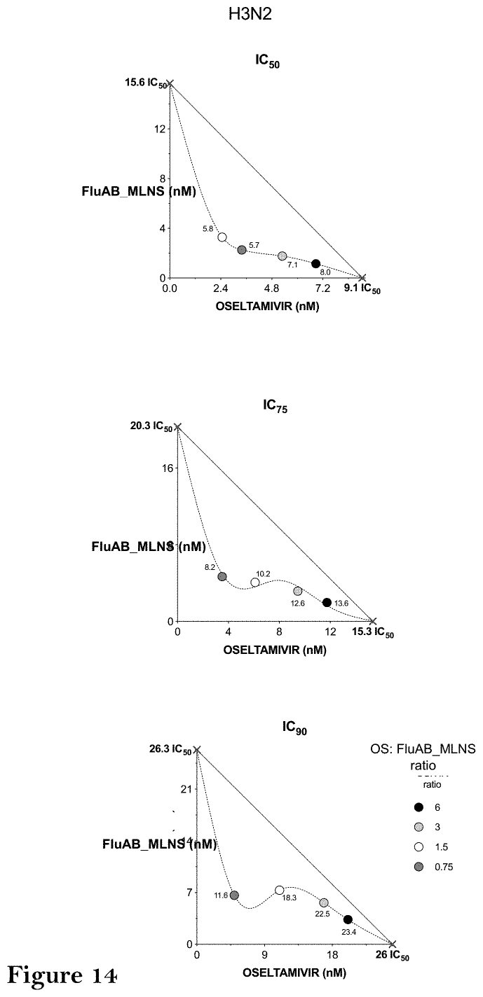

The same data can be alternatively described with isobolograms plots, which compare the equipotent concentrations of both the single and combined drugs. As shown in FIGS. 13 and 14, the distribution of the IC₅₀, IC₇₅, and IC₉₀ values for the three different combination ratios is by far below the isobole lines connecting the respective IC₅₀, IC₇₅, and IC₉₀ of the single drugs tested, both for H1 (FIG. 13) and H3 (FIG. 14), indicating consistent synergy (while an additive and antagonism would generate equipotency points localized either onto or over the single-drug isobole, respectively).

Combination of FluAB_MLNS and Zanamivir

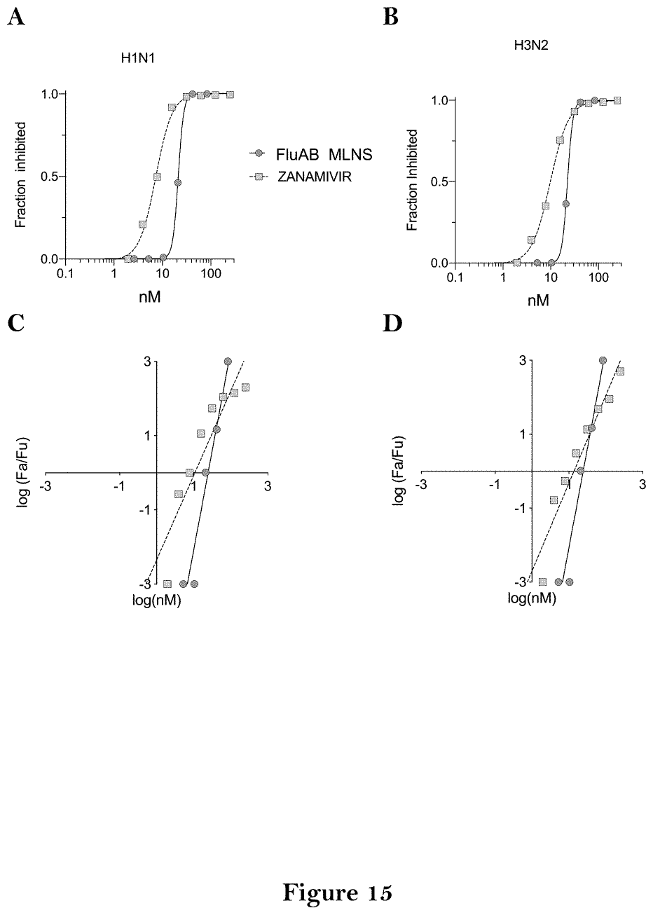

The relative efficacy of FluAB_MLNS and zanamivir to neutralize influenza A viruses was also compared in vitro on two viral serotype representatives for both H3N2 and H1N1 strains. As shown in FIG. 15, both compounds, tested separately, were dose-dependently capable to fully inhibit cell infection, when independently exposed together with H3N3 and H1N1 virus. The relative calculated IC₅₀ values were 23.1-24.4 nM for FluAB_MLNS and 10.7-13.7 nM for zanamivir.

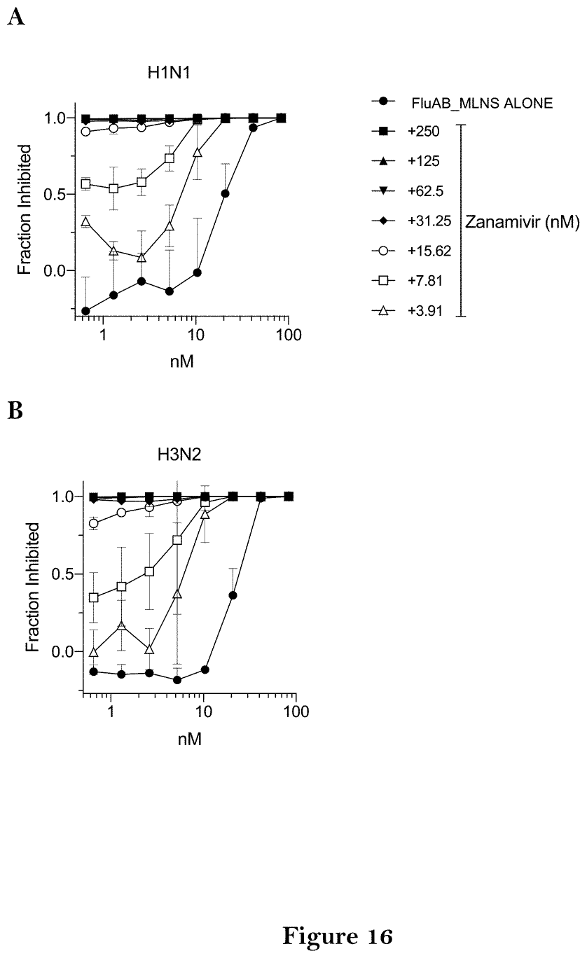

For the combined effect of FluAB_MLNS and zanamivir FIG. 16 shows that, similarly to oseltamivir, zanamivir greatly enhances the inhibitory capacity of FluAB_MLNS both against H1 and H3 viruses.

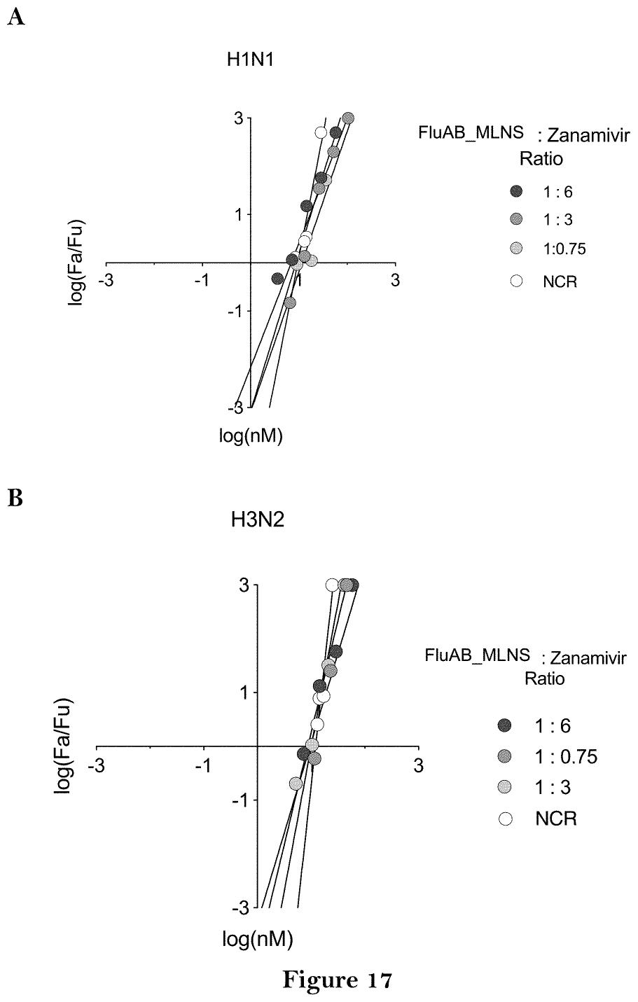

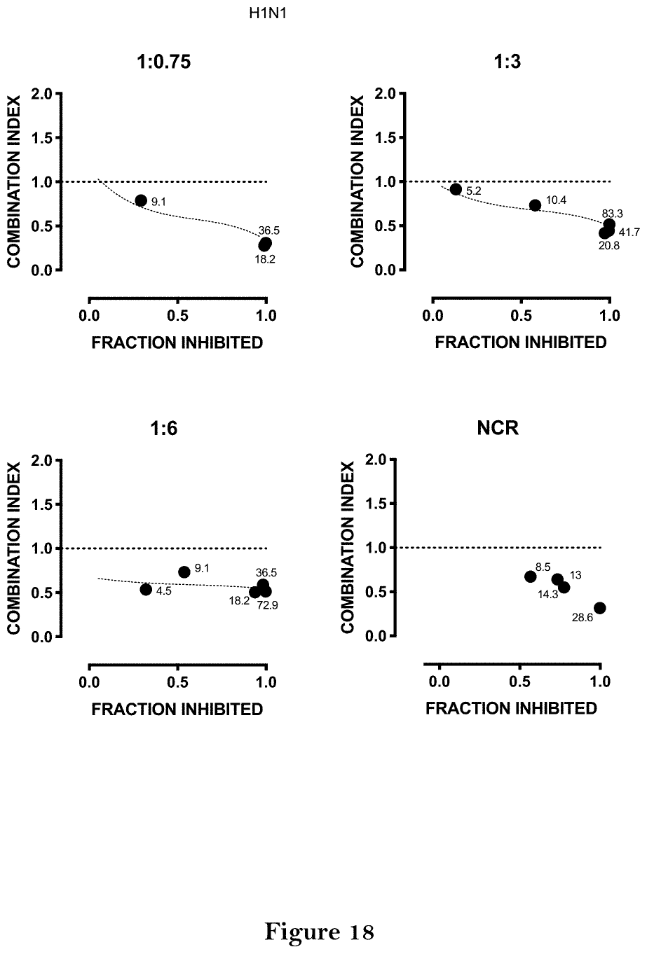

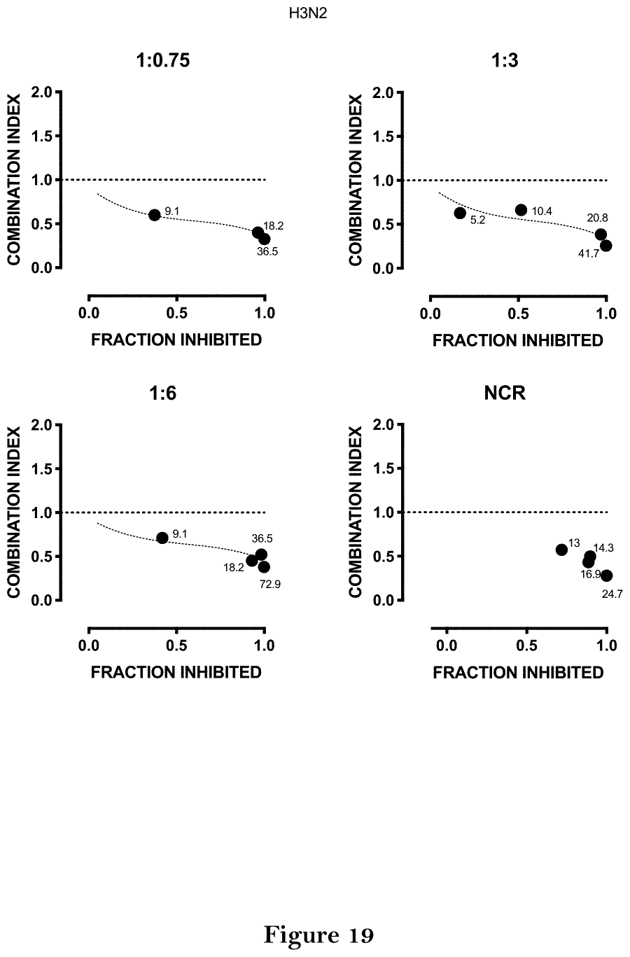

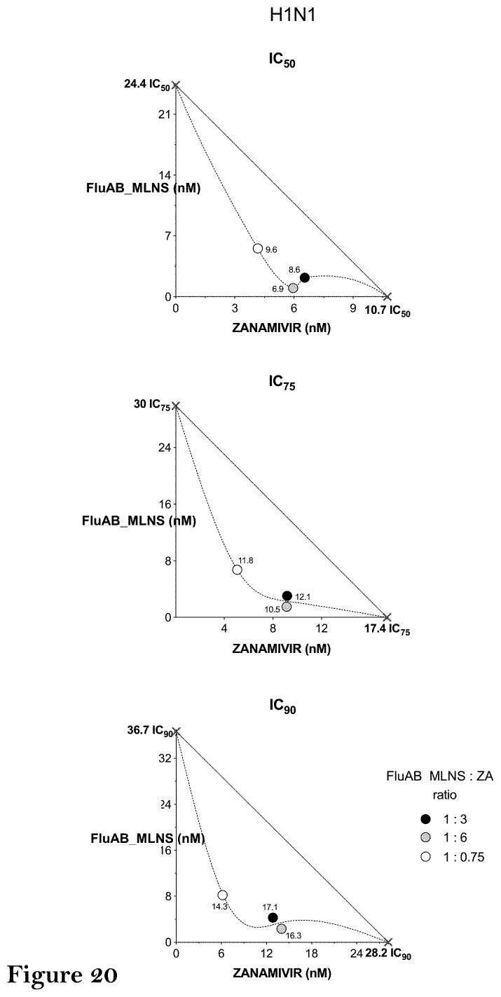

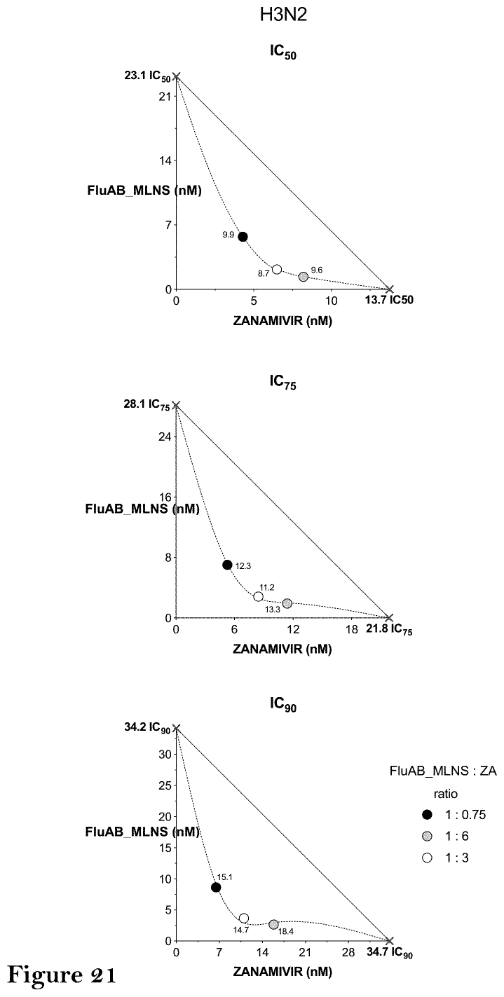

The quantification of the synergic effect was similarly computed with CompuSyn and the median effect principle as described above. The median effect plots for the combined effects of FluAB_MLNS and zanamivir are shown in FIG. 17. The calculated CI for FluAB_MLNS and zanamivir is shown in FIGS. 18 and 19 and clearly indicates a synergistic effect between the two drugs, both with H1 (FIG. 18) and H3 (FIG. 19) viruses, as indicated by the values lower than 1 for all the experimental point tested. Consistently, with both viral strains, the isobolograms denote a strong synergistic effect across the IC₅₀, IC₇₅, and IC₉₀ values (shown in FIGS. 20 and 21), which are all significantly below the IC values with the single drug.

Combination of FluAB_MLNS and Baloxavir Marboxil

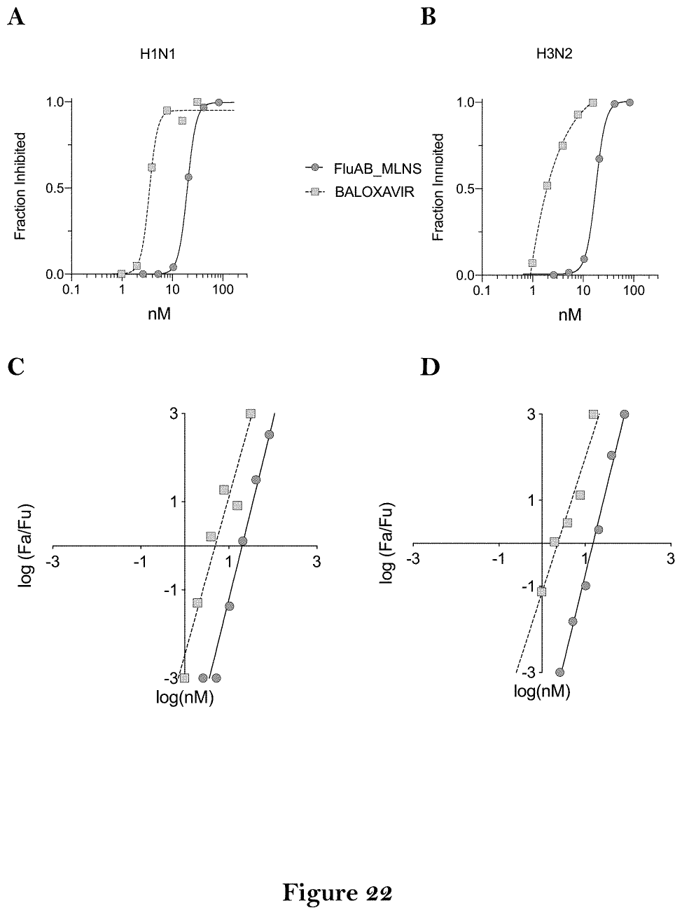

The recently approved endonuclease inhibitor baloxavir marboxil was initially compared with FluAB_MLNS alone on both H1 and H3 strains, similarly as described above for oseltamivir and zanamivir. Results are shown in FIG. 22. The relative calculated IC₅₀ values were 20.1-15.4 nM for FluAB_MLNS and 4.9-2.3 nM for baloxavir marboxil.

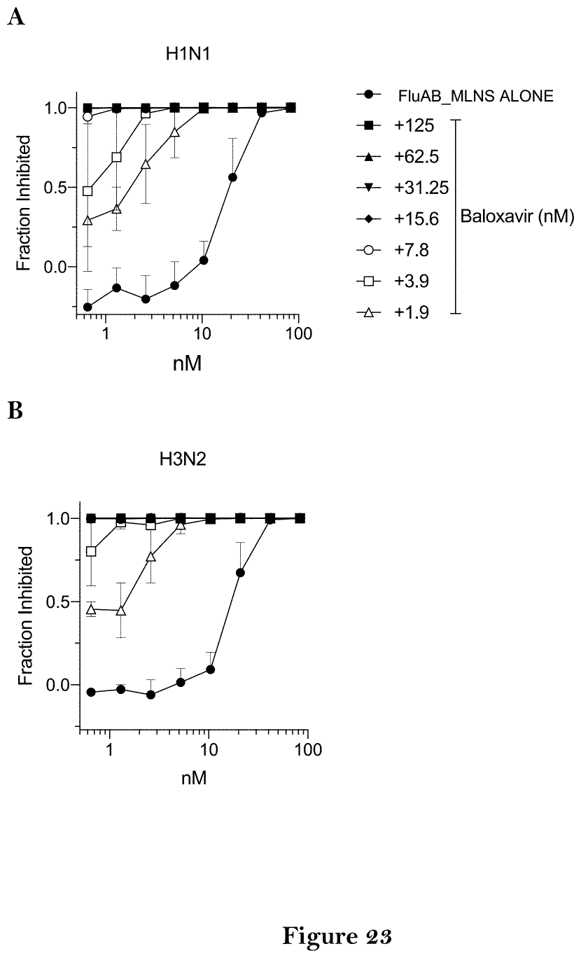

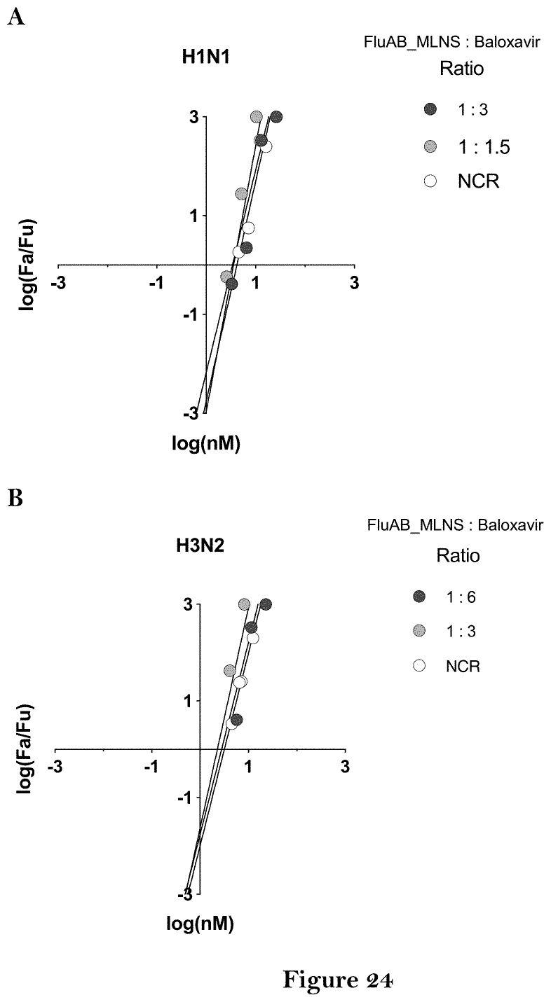

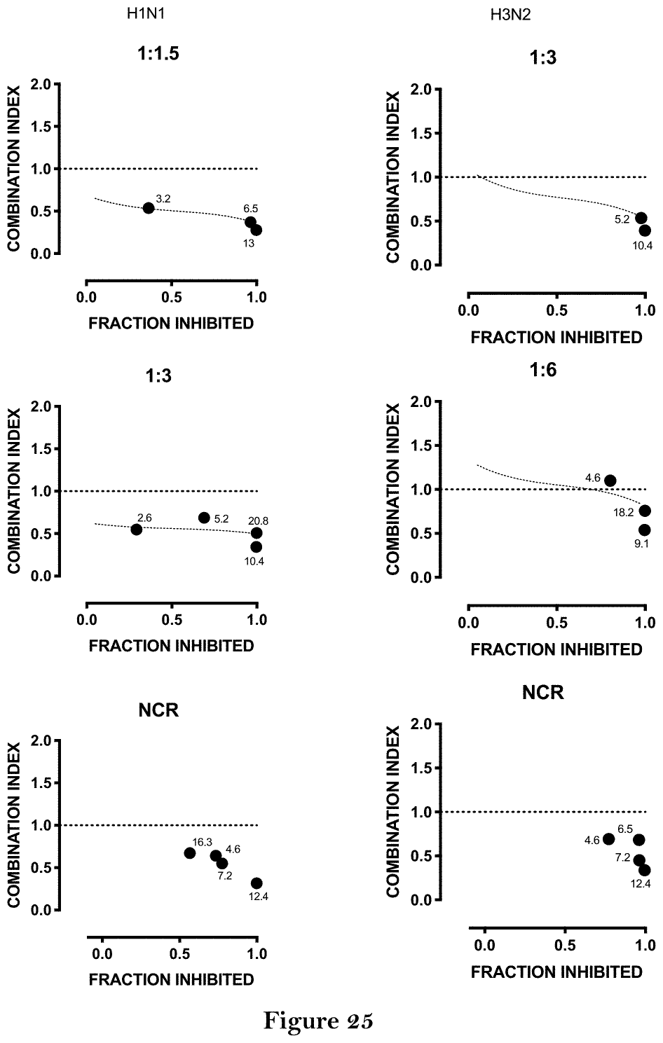

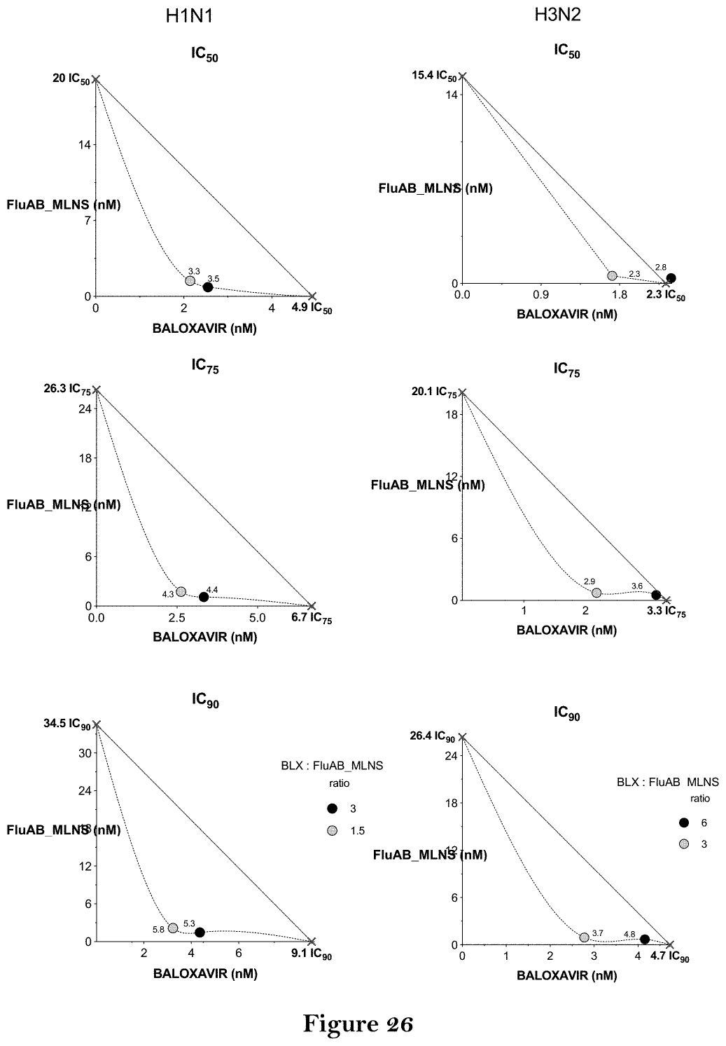

Although Baloxavir has a different mechanism of action in inhibiting viral replication compared to the NA inhibitors, the drug is still able to strongly enhance the inhibitory capacity of FluAB_MLNS, clearly indicating a synergistic effect (FIG. 23). The inhibition data obtained with the different combination ratios were used to compute and plot the median effect with CompuSyn software and calculate the type of drug-drug interaction as described above (FIG. 24). The calculated CI for FluAB_MLNS and baloxavir marboxil (FIG. 25) clearly indicate a synergistic effect between the two drugs, both with H1 and H3 viruses, as indicated by the values lower than 1 for the majority of the experimental points tested. The isobolograms denote a robust and complete synergistic effect across the IC₅₀, IC₇₅, and IC₉₀ values (FIG. 26).

In summary, the neutralization capacity of FluAB_MLNS against both H1 and H3 strains is synergistically enhanced by different antivirals, namely, the NA inhibitors oseltamivir and zanamivir as well as the endonuclease inhibitor baloxavir-marboxil.

### Example 7: Binding to Human FcRn at Different pHs

FluAB_wt and FluAB_MLNS were compared side by side for their ability to bind to neonatal Fc receptor (FcRn) using biolayer interferometry (BLI).

To this end, binding of FluAB_wt and FluAB_MLNS to human FcRn was measured on an Octet RED96 instrument (biolayer interferometry, BLI, ForteBio). Biosensors coated with anti-human Fab-CH1 were pre-hydrated in kinetic buffer for 10 min at RT. Then, human mAb (FluAB_wt or FluAB_MLNS) was loaded at 1 μg/ml in kinetics buffer at pH 7.4 for 30 minutes onto the Biosensors. The baseline was measured in kinetics buffer (Sterile filtered 0.01% endotoxin-free bovine serum albumin, 0.002% Tween-20 (Polysorbate 20), 0.005% NaN3 in PBS) at pH=7.4 or pH=6.0 for 4 minutes. Human mAb-loaded sensors were then exposed for 7 minutes to a solution of human FcRn at 1 μg/ml in kinetics buffer at pH=7.4 or pH=6.0 to measure association of FcRn-mAb in different milieus (on rate). Dissociation was then measured in kinetics buffer at the same pH for additional 5 minutes (off rate). All steps were performed while stirring at 1000 rpm at 30° C. Association and dissociation profiles were measured in real time as change in the interference patterns.

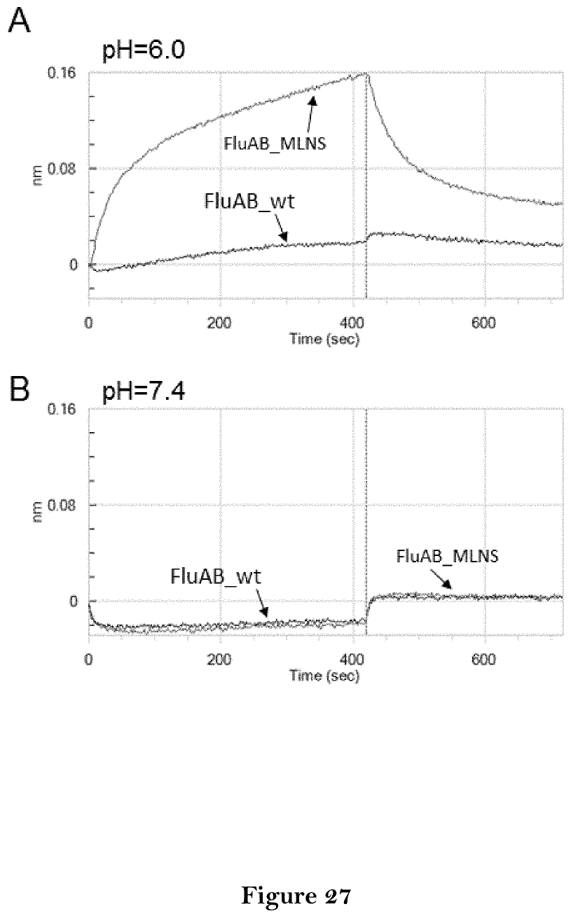

As shown in FIG. 27, FluAB_MLNS bound human FcRn with higher affinity compared to FluAB_wt at acidic pH (pH 6.0), while neither FluAB_MLNS nor FluAB_wt binds FcRn at neutral pH (pH 7.4).

### Example 8: Characterization of Polymorphisms Identified in the Antibody's Extended Epitope

Historical polymorphisms in the extended epitope were evaluated for their impact on neutralization activity of FluAB_MLNS using viruses generated by reverse genetics with H1 HA or H3 HA on a A/Puerto Rico/8/34 (PR8) background.

Single nucleotide polymorphisms were introduced into PR8 H1 HA or A/Aichi/2/68 (Aichi) HA pHW2000 plasmids using site-directed mutagenesis. Recombinant influenza A virus were rescued with associated H1 or H3 HA on a PR8 backbone using standard methods (e.g., as described in Erich Hoffmann, Gabriele Neumann, Yoshihiro Kawaoka, Gerd Hobom, Robert G. Webster, 2000, A DNA transfection system for generation of influenza A virus from eight plasmids. Proceedings of the National Academy of Sciences May 2000, 97 (11): 6108-6113; doi: 101073/pnas.100133697).

Neutralization activity was evaluated in MDCK cells using standard methods. For example, neutralization activity may be evaluated in MDCK cells, e.g. in 96 well plates. To this end, MCDK cells may be seeded at 30,000 cells/well 24 hours prior to infection. Antibody FluAB_MLNS may be incubated with virus for 1 hour at 37° C. prior to addition to MDCK cells. To this end, 1:2.5 9-point serial dilutions of FluAB_MLNS may be created in infection media and each dilution may be tested in triplicate (e.g., 50 μg/mL-0.03 μg/mL final concentration) and may be incubated with 120 TCID₅₀ of virus for 1 hour at 37° C. MDCK cells may be washed twice with PBS, 100 μl/well of virus:antibody solution may be added, and cells may be incubated for 4 hours at 37° C. After 4 hours, an additional 100 μl/well of infection media may be added to cells. After 72 hours of incubation at 37° C., viral RNA may be extracted and measured by qRT-PCR, e.g. using WHO primers (World Health Organization. CDC protocol of real-time RT-PCR for influenza A H1N1. Apr. 28, 2009). The IC50 is expressed as the antibody concentration in μg/mL that reduces 50% of virus replication and may be calculated using a non-linear 4-parameter logistic fit curve of data normalized to control wells (no virus and virus alone).

The neutralization activity of FluAB_MLNS to H1 and H3 HA polymorphisms in the extended epitope is shown in Table 4 below.

TABLE 4

| | Amino | FluAB_MLNS | |
|---|---|---|---|
| | Acid | Geomean | Fold change |
| | Changes | Neutralization | relative to |
| Virus | in HA | IC₅₀ (μg/mL) | WT virus |
| PR8: Aichi HA wt | wild type | 5.6 | NA |
| PR8: Aichi HA P11S | P11S | 9.5 | 1.7 |
| PR8: Aichi HA D46N | D46N | 3.3 | 0.6 |
| PR8: Aichi HA N49T | N49T | 5.0 | 0.9 |
| PR8 wt | wild type | 4.7 | NA |
| PR8 HA N146D | N146D | 5.5 | 1.2 |

Aichi = A/Aichi/2/68;
Geomean = geometric mean;
HA = hemagglutinin;
NA = not applicable;
PR8 = A/Puerto Rico/8/34 H1N1;
wt = wild type

For viruses encoding H3 HA, FluAB_MLNS neutralized viruses with mutations HA1 P11S, HA2 D46N, or HA2 N49T with IC₅₀ values similar to wild type virus (<2-fold change in IC₅₀ relative to wild type virus). For viruses encoding H1 HA, FluAB_MLNS neutralized viruses with encoding HA2 N146D with IC₅₀ values similar to wild type virus (<2-fold change in IC₅₀ relative to wild type virus). Additionally, the PR8 wild type strain used encoded the HA2 polymorphism L38Q and D46N and was neutralized with an IC₅₀ value of 4.7 μg/mL by FluAB_MLNS. Overall, all polymorphisms evaluated resulted in IC₅₀ fold changes of <2 relative to the wild type virus for FluAB_MLNS. In summary, FluAB_MLNS effectively neutralized all evaluated historical polymorphisms in the extended epitope (H3 HA: HA1 P11S, HA2 D46N, or HA2 N49T; H1 HA: N146D).

### Example 9: Anti-Drug Antibody Response in Tg32 Mice

With regard to the M428L/N434S mutation, recently concerns were raised that said mutation increases immunogenicity of antibodies comprising this mutation (Brian C. Mackness, Julie A. Jaworski, Ekaterina Boudanova, Anna Park, Delphine Valente, Christine Mauriac, Olivier Pasquier, Thorsten Schmidt, Mostafa Kabiri, Abdullah Kandira, Katarina Radošević & Huawei Qiu (2019) Antibody Fc engineering for enhanced neonatal Fc receptor binding and prolonged circulation half-life, mAbs, 11:7, 1276-1288; Maeda A, Iwayanagi Y, Haraya K, et al. Identification of human IgG1 variant with enhanced FcRn binding and without increased binding to rheumatoid factor autoantibody. MAbs. 2017; 9(5):844-853).

To assess immunogenicity, in particular the anti-drug response (anti-drug antibodies; ADA), of antibody FluAB_MLNS in comparison to its parental antibody FluAB_wt, two separate groups (n=5) of TG32 mice (transgenic for the human FcRn) were injected i.v. with 5 mg/kg of either FluAB-MLNS or FluAB_wt monoclonal antibodies. To evaluate the circulating levels of the injected mAbs, blood samples were then obtained at different time points. Samples taken at day 14 and 21 post injection were used to evaluate, by specific ELISA, the anti-drug antibody (ADA) response against the injected human monoclonals.

Briefly, purified FluAB_wt and FluAB_MLNS monoclonal antibodies were coated on 96-well plates at 2 μg/ml. After blocking, the sera from treated animals obtained 14 and 21 days post injection, diluted 1:180, were incubated 1.5 h at room temperature (RT). After washings, peroxidase-labeled goat anti-mouse IgG F(ab′)2 fragment (0.16 μg/ml) was added to plates and incubated 1.5 h at RT. ADA IgG (murine antibodies against the injected antibodies FluAB_wt and FluAB_MLNS) were then revealed with the appropriate substrate and read with a spectrophotometer. Data shown are the OD values (450 nm) obtained in each individual serum (n=5/group) collected 14 and 21 days after the antibody i.v. administration. Sera from naïve Tg32 mice (ctrl) were used as negative control.

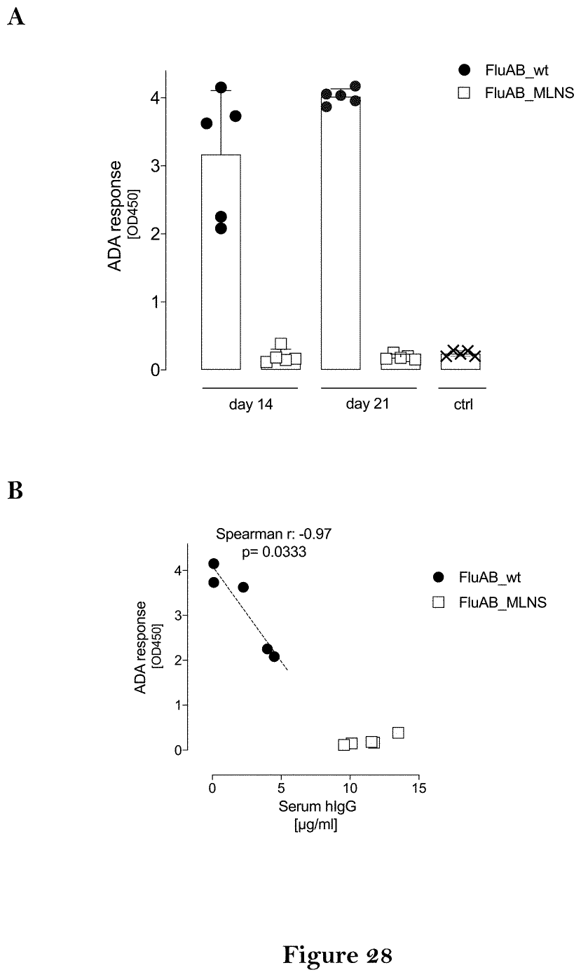

Results are shown in FIG. 28. Surprisingly, the signal of murine serum IgG reacting against the FluAB-MLNS antibody was very low and corresponding to the signal detected in control, non-injected animals, while the ADA response measured in the serum of mice injected with FluAB_wt was, instead, significantly high, both 14 and 21 day after i.v. injection (FIG. 28A). In addition, the levels of ADA measured at day 14 post injection significantly and inversely correlated with the levels of circulating FluAB_wt (serum FluAB_wt levels decreased due to murine antibodies against FluAB_wt), while the levels of circulating FluAB-MLNS measured at the same time were indeed much higher and homogeneous (FIG. 28B).

In summary, these data indicate that surprisingly the anti-drug response (anti-drug antibodies; ADA), and, thus, the immunogenicity of FluAB_MLNS was decreased compared to FluAB_wt.

### Example 10: Anti-Drug Antibody Response and Immunogenicity after s.c. Administration

To further confirm this surprising finding in more immunogenic settings, separate groups of TG32 mice (n=5) were injected with either FluAB-MLNS or FluAB_wt (5 mg/kg) subcutaneously (s.c.), which is generally considered a more immunogenic route of administration. Three weeks after s.c. administration, the levels of anti-drug antibodies were measured in the serum by mouse anti-drug specific ELISA (as described above in Example 9) in the serum of mice injected s.c. with either FluAB_wt or FLuAB_MLNS. As negative control, a pool of 10 sera from naïve, untreated animals was used.

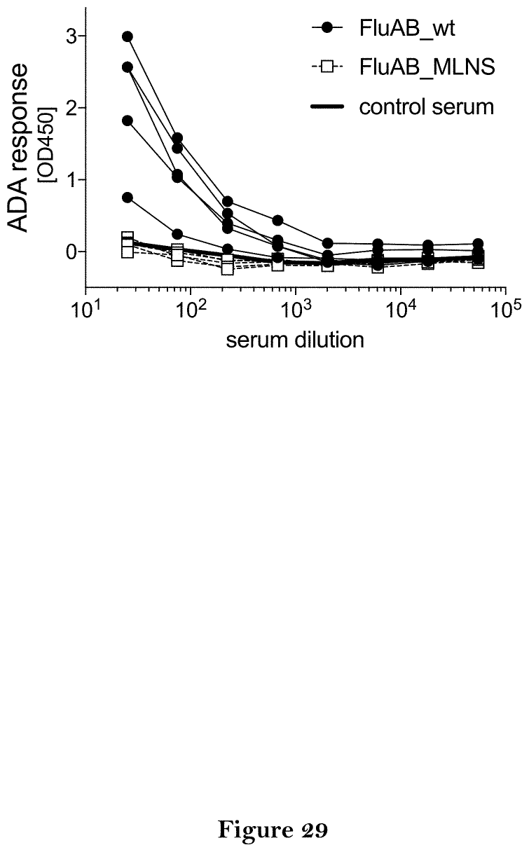

Results are shown in FIG. 29. Despite the more immunogenic settings, animals treated s.c. with FluAB-MLNS still did not mount a humoral immunogenic response, as confirmed by the serum titer of anti-hIgG antibodies that was overlapping to the one detected in the serum of non-injected control animals. Conversely, the ADA titer in animals treated with FluAB_wt was clearly positive and measurable in all treated animals. An inverse correlation between the circulating levels of injected antibody and anti hIgG endogenous response was detected in the sera of mice injected with FluAB_wt only (not shown).

These data surprisingly show that antibody FluAB_MLNS exhibits less immunogenicity as compared to its parental antibody FluAB_wt.

TABLE OF SEQUENCES AND SEQ ID NUMBERS (SEQUENCE LISTING):

| | | |
|---|---|---|
| SEQ ID NO | Sequence | Remarks |

FluAB_MLNS

| | | |
|---|---|---|
| SEQ ID NO: 1 | SYNAVWN | CDRH1 |
| SEQ ID NO: 2 | RTYYRSGWYNDYAESVKS | CDRH2 |
| SEQ ID NO: 3 | SGHITVFGVNVDAFDM | CDRH3 |
| SEQ ID NO: 4 | RTSQSLSSYTH | CDRL1 |
| SEQ ID NO: 5 | AASSRGS | CDRL2 |
| SEQ ID NO: 6 | QQSRT | CDRL3 |
| SEQ ID NO: 7 | QVQLQQSGPGLVKPSQTLSLTCAISGDSVSSYNAVWN | VH |
| | WIRQSPSRGLEWLGRTYYRSGWYNDYAESVKSRITINPD | |
| | TSKNQFSLQLNSVTPEDTAVYYCARSGHITVFGVNVDAF | |
| | DMWGQGTMVTVSS | |
| SEQ ID NO: 8 | DIQMTQSPSSLSASVGDRVTITCRTSQSLSSYTHWYQQK | VL |
| | PGKAPKLLIYAASSRGSGVPSRFSGSGSGTDFTLTISSLQP | |
| | EDFATYYCQQSRTFGQGTKVEIK | |
| SEQ ID NO: 9 | QVQLQQSGPGLVKPSQTLSLTCAISGDSVSSYNAVWNW | Heavy chain |
| | IRQSPSRGLEWLGRTYYRSGWYNDYAESVKSRITINPDTS | |
| | KNQFSLQLNSVTPEDTAVYYCARSGHITVFGVNVDAFD | |
| | MWGQGTMVTVSSASTKGPSVFPLAPSSKSTSGGTAALG | |
| | CLVKDYFPEPVTVSWNSGALTSGVHTFPAVLQSSGLYSLS | |
| | SVVTVPSSSLGTQTYICNVNHKPSNTKVDKRVEPKSCDKT | |
| | HTCPPCPAPELLGGPSVFLFPPKPKDTLMISRTPEVTCVV | |
| | VDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSREEMTKNQVSLTCLVKGFYPSDIAVE | |
| | WESNGQPENNYKTTPPVLDSDGSFFLYSKLTVDKSRWQ | |
| | QGNVFSCSVLHEALHSHYTQKSLSLSPGK | |
| SEQ ID NO: 10 | DIQMTQSPSSLSASVGDRVTITCRTSQSLSSYTHWYQQK | Light chain |
| | PGKAPKLLIYAASSRGSGVPSRFSGSGSGTDFTLTISSLQP | |
| | EDFATYYCQQSRTFGQGTKVEIKRTVAAPSVFIFPPSDEQ | |
| | LKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |

FluAB_wt

| | | |
|---|---|---|
| SEQ ID NO: 11 | QVQLQQSGPGLVKPSQTLSLTCAISGDSVSSYNAVWN | Heavy chain |
| | WIRQSPSRGLEWLGRTYYRSGWYNDYAESVKSRITINPD | |
| | TSKNQFSLQLNSVTPEDTAVYYCARSGHITVFGVNVDAF | |
| | DMWGQGTMVTVSSASTKGPSVFPLAPSSKSTSGGTAA | |
| | LGCLVKDYFPEPVTVSWNSGALTSGVHTFPAVLQSSGLY | |
| | SLSSVVTVPSSSLGTQTYICNVNHKPSNTKVDKRVEPKSC | |
| | DKTHTCPPCPAPELLGGPSVFLFPPKPKDTLMISRTPEVT | |
| | CVVVDVSHEDPEVKFNWYVDGVEVHNAKTKPREEQYN | |
| | STYRVVSVLTVLHQDWLNGKEYKCKVSNKALPAPIEKTIS | |
| | KAKGQPREPQVYTLPPSREEMTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |

FluAB_MLNS nucleic acid sequences

| | | |
|---|---|---|
| SEQ ID NO: 12 | CAAGTTCAGCTGCAGCAGAGCGGCCCCGGTCTGGTGA | FluAB_MLNS VH nuc |
| | AGCCTAGCCAGACTCTGTCTTTAACTTGCGCCATCTCC | |
| | GGCGACAGCGTGAGCAGCTACAACGCCGTCTGGAACT | |
| | GGATTCGTCAGAGCCCTAGCAGAGGTTTAGAGTGGCT | |
| | GGGTCGTACTTACTATCGTTCCGGCTGGTACAACGACT | |
| | ACGCCGAGAGCGTGAAGTCTCGTATCACTATCAACCC | |
| | CGATACTAGCAAGAACCAGTTCTCTTTACAGCTGAACA | |
| | GCGTGACTCCCGAAGACACTGCCGTGTACTACTGCGC | |
| | TCGTAGCGGCCACATCACTGTGTTCGGCGTGAATGTG | |
| | GACGCCTTCGACATGTGGGGCCAAGGTACTATGGTCA | |
| | CTGTGAGCAGC | |
| SEQ ID NO: 13 | GACATCCAGATGACTCAGAGCCCTTCCTCTTTAAGCGC | FluAB_MLNS VL nuc |
| | TAGCGTGGGCGATAGGGTCACTATCACTTGTCGTACT | |
| | AGCCAGTCTTTAAGCTCCTACACTCACTGGTACCAGCA | |
| | GAAGCCCGGTAAGGCCCCTAAGCTGCTGATCTACGCT | |
| | GCCAGCAGCAGAGGCAGCGGAGTGCCTAGCAGATTT | |
| | AGCGGCAGCGGTAGCGGCACTGACTTCACTCTGACAA | |
| | TCAGCTCTTTACAGCCCGAAGACTTCGCCACTTACTAC | |
| | TGCCAGCAGTCTCGTACTTTCGGCCAAGGTACTAAGGT | |
| | GGAGATCAAG | |
| SEQ ID NO: 14 | CAAGTTCAGCTGCAGCAGAGCGGCCCCGGTCTGGTGA | FluAB_MLNS heavy |
| | AGCCTAGCCAGACTCTGTCTTTAACTTGCGCCATCTCC | chain nuc |
| | GGCGACAGCGTGAGCAGCTACAACGCCGTCTGGAACT | |
| | GGATTCGTCAGAGCCCTAGCAGAGGTTTAGAGTGGCT | |
| | GGGTCGTACTTACTATCGTTCCGGCTGGTACAACGACT | |
| | ACGCCGAGAGCGTGAAGTCTCGTATCACTATCAACCC | |
| | CGATACTAGCAAGAACCAGTTCTCTTTACAGCTGAACA | |
| | GCGTGACTCCCGAAGACACTGCCGTGTACTACTGCGC | |
| | TCGTAGCGGCCACATCACTGTGTTCGGCGTGAATGTG | |
| | GACGCCTTCGACATGTGGGGCCAAGGTACTATGGTCA | |
| | CTGTGAGCAGCGCTAGCACCAAGGGCCCATCGGTCTT | |
| | CCCCCTGGCACCCTCCTCCAAGAGCACCTCTGGGGGC | |
| | ACAGCGGCCCTGGGCTGCCTGGTCAAGGACTACTTCC | |
| | CCGAACCGGTGACGGTGTCGTGGAACTCAGGCGCCCT | |
| | GACCAGCGGCGTGCACACCTTCCCGGCCGTCCTACAG | |
| | TCCTCAGGACTCTACTCCCTCAGCAGCGTGGTGACCGT | |
| | GCCCTCCAGCAGCTTGGGCACCCAGACCTACATCTGCA | |
| | ACGTGAATCACAAGCCCAGCAACACCAAGGTGGACAA | |
| | GCGGGTTGAGCCCAAATCTTGTGACAAAACTCACACA | |
| | TGCCCACCGTGCCCAGCACCTGAACTCCTGGCGGGAC | |
| | CGTCAGTCTTCCTCTTCCCCCCAAAACCCAAGGACACC | |
| | CTCATGATCTCCCGGACCCCTGAGGTCACATGCGTGGT | |
| | GGTGGACGTGAGCCACGAAGACCCTGAGGTCAAGTTC | |
| | AACTGGTACGTGGACGGCGTGGAGGTGCATAATGCC | |
| | AAGACAAAGCCGCGGGAGGAGCAGTACAACAGCACG | |
| | TACCGTGTGGTCAGCGTCCTCACCGTCCTGCACCAGGA | |
| | CTGGCTGAATGGCAAGGAGTACAAGTGCAAGGTCTCC | |
| | AACAAAGCCCTCCCACTCCCCGAAGAGAAAACCATCTC | |
| | CAAAGCCAAAGGGCAGCCCCGAGAACCACAGGTGTA | |
| | CACCCTGCCCCCATCCCGGGAGGAGATGACCAAGAAC | |
| | CAGGTCAGCCTGACCTGCCTGGTCAAAGGCTTCTATCC | |
| | CAGCGACATCGCCGTGGAGTGGGAGAGCAATGGGCA | |
| | GCCGGAGAACAACTACAAGACCACGCCTCCCGTGCTG | |
| | GACTCCGACGGCTCCTTCTTCCTCTACAGCAAGCTCAC | |
| | CGTGGACAAGAGCAGGTGGCAGCAGGGGAACGTCTT | |
| | CTCATGCTCCGTGCTGCATGAGGCTCTGCACAGCCACT | |
| | ACACGCAGAAGAGCCTCTCCCTGTCTCCGGGTAAA | |
| SEQ ID NO: 15 | GACATCCAGATGACTCAGAGCCCTTCCTCTTTAAGCGC | FluAB_MLNS light |
| | TAGCGTGGGCGATAGGGTCACTATCACTTGTCGTACT | chain nuc |
| | AGCCAGTCTTTAAGCTCCTACACTCACTGGTACCAGCA | |
| | GAAGCCCGGTAAGGCCCCTAAGCTGCTGATCTACGCT | |
| | GCCAGCAGCAGAGGCAGCGGAGTGCCTAGCAGATTT | |
| | AGCGGCAGCGGTAGCGGCACTGACTTCACTCTGACAA | |
| | TCAGCTCTTTACAGCCCGAAGACTTCGCCACTTACTAC | |
| | TGCCAGCAGTCTCGTACTTTCGGCCAAGGTACTAAGGT | |
| | GGAGATCAAGCGTACGGTGGCTGCACCATCTGTCTTC | |
| | ATCTTCCCGCCATCTGATGAGCAGTTGAAATCTGGAAC | |
| | TGCCTCTGTTGTGTGCCTGCTGAATAACTTCTATCCCA | |
| | GAGAGGCCAAAGTACAGTGGAAGGTGGATAACGCCC | |
| | TCCAATCGGGTAACTCCCAGGAGAGTGTCACAGAGCA | |
| | GGACAGCAAGGACAGCACCTACAGCCTCAGCAGCACC | |
| | CTGACGCTGAGCAAAGCAGACTACGAGAAACACAAA | |
| | GTCTACGCCTGCGAAGTCACCCATCAGGGCCTGAGCT | |
| | CGCCCGTCACAAAGAGCTTCAACAGGGGAGAGTGT | |

1. An antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S in the constant region of the heavy chain.

2. The antibody of claim 1, wherein the antibody binds to hemagglutinin of an influenza A virus.

3. The antibody of claim 1 or 2, wherein the antibody neutralizes infection with an influenza A virus.

4. The antibody of claim 3, wherein the antibody neutralizes influenza A infection at a dose, which does not exceed half of the dose required for neutralization of influenza A with a comparative antibody, which differs from said antibody only in that it does not contain the mutations M428L and N434S in the constant region of the heavy chain.

5. The antibody of claim 4, wherein the dose does not exceed one third of the dose required for neutralization of influenza A with said comparative antibody.

6. The antibody of claim 4 or 5, wherein the dose does not exceed one fifth of the dose required for neutralization of influenza A with said comparative antibody.

7. The antibody of any one of the previous claims, wherein the antibody neutralizes polymorphisms HA1 P11S, HA2 D46N, and/or HA2 N49T of H3 HA; and/or polymorphism N146D of H1 HA.

8. The antibody of any one of claim 7, wherein the antibody neutralizes polymorphisms HA1 P11S, HA2 D46N, and/or HA2 N49T of H3 HA; and/or polymorphism N146D of H1 HA with IC₅₀ fold changes of <2 relative to HA of the wild type virus.

9. The antibody of any one of the previous claims, wherein the antibody elicits a decreased anti-drug antibody response as compared to a comparative antibody differing from said antibody only in that it does not contain the mutations M428L and N434S in the constant region of the heavy chain.

10. The antibody of any one of the previous claims, wherein the antibody exhibits less immunogenicity as compared to a comparative antibody differing from said antibody only in that it does not contain the mutations M428L and N434S in the constant region of the heavy chain.

11. The antibody of any one of the previous claims, wherein the antibody is a human antibody.

12. The antibody of any one of the previous claims, wherein the antibody is a monoclonal antibody.

13. The antibody of any one of the previous claims, wherein the antibody is of the IgG type.

14. The antibody of claim 13, wherein the antibody is of the IgG1 type.

15. The antibody of any one of the previous claims, wherein the light chain of the antibody is a kappa light chain.

16. The antibody of any one of the previous claims, wherein the antibody comprises a heavy chain variable region comprising an amino acid sequence having at least 70% identity to SEQ ID NO: 7 and a light chain variable region comprising the amino acid sequence having at least 70% identity to SEQ ID NO: 8, wherein the CDR sequences as defined in claim 1 are maintained.

17. The antibody of any one of the previous claims, wherein the antibody comprises a heavy chain variable region comprising an amino acid sequence having at least 75% identity to SEQ ID NO: 7 and a light chain variable region comprising the amino acid sequence having at least 75% identity to SEQ ID NO: 8, wherein the CDR sequences as defined in claim 1 are maintained.

18. The antibody of any one of the previous claims, wherein the antibody comprises a heavy chain variable region comprising an amino acid sequence having at least 80% identity to SEQ ID NO: 7 and a light chain variable region comprising the amino acid sequence having at least 80% identity to SEQ ID NO: 8, wherein the CDR sequences as defined in claim 1 are maintained.

19. The antibody of any one of the previous claims, wherein the antibody comprises a heavy chain variable region comprising an amino acid sequence having at least 85% identity to SEQ ID NO: 7 and a light chain variable region comprising the amino acid sequence having at least 85% identity to SEQ ID NO: 8, wherein the CDR sequences as defined in claim 1 are maintained.

20. The antibody of any one of the previous claims, wherein the antibody comprises a heavy chain variable region comprising an amino acid sequence having at least 90% identity to SEQ ID NO: 7 and a light chain variable region comprising the amino acid sequence having at least 90% identity to SEQ ID NO: 8, wherein the CDR sequences as defined in claim 1 are maintained.

21. The antibody of any one of the previous claims, wherein the antibody comprises a heavy chain variable region comprising an amino acid sequence having at least 95% identity to SEQ ID NO: 7 and a light chain variable region comprising the amino acid sequence having at least 95% identity to SEQ ID NO: 8, wherein the CDR sequences as defined in claim 1 are maintained.

22. The antibody of any one of the previous claims, wherein the antibody comprises a heavy chain variable region comprising an amino acid sequence as set forth in SEQ ID NO: 7 and a light chain variable region comprising the amino acid sequence as set forth in SEQ ID NO: 8, wherein the CDR sequences as defined in claim 1 are maintained.

23. The antibody of any one of the previous claims, wherein the CH3 region of the antibody does not comprise any further mutation in addition to M428L and N434S.

24. The antibody of any one of the previous claims, wherein the Fc region of the antibody does not comprise any further mutation in addition to M428L and N434S.

25. The antibody of any one of the previous claims, wherein the antibody comprises a heavy chain comprising an amino acid sequence as set forth in SEQ ID NO: 9 and a light chain comprising an amino acid sequence as set forth in SEQ ID NO: 10.

26. The antibody of any one of the previous claims, wherein the antibody has a heavy chain consisting of an amino acid sequence as set forth in SEQ ID NO: 9 and a light chain consisting of an amino acid sequence as set forth in SEQ ID NO: 10.

27. The antibody of any one of the previous claims for use in prophylaxis or treatment of infection with influenza A virus.

28. The antibody for use according to claim 27, wherein the antibody is administered prophylactically.

29. The antibody for use according to claim 27 or 28, wherein the antibody is administered at a dose which does not exceed half of the dose required for prophylaxis or treatment of influenza A with a comparative antibody, which differs from said antibody only in that it does not contain the mutations M428L and N434S in the constant region of the heavy chain.

30. The antibody for use according to claim 29, wherein the dose does not exceed one third of the dose required for prophylaxis or treatment of influenza A with said comparative antibody.

31. The antibody for use according to claim 29, wherein the dose does not exceed one quarter of the dose required for prophylaxis or treatment of influenza A with said comparative antibody.

32. The antibody for use according to claim 29, wherein the dose does not exceed one fifth of the dose required for prophylaxis or treatment of influenza A with said comparative antibody.

33. The antibody for use according to claim 29, wherein the dose does not exceed one sixth of the dose required for prophylaxis or treatment of influenza A with said comparative antibody.

34. The antibody for use according to claim 29, wherein the dose does not exceed one seventh of the dose required for prophylaxis or treatment of influenza A with said comparative antibody.

35. The antibody for use according to claim 29, wherein the dose does not exceed one eighth of the dose required for prophylaxis or treatment of influenza A with said comparative antibody.

36. The antibody for use according to claim 29, wherein the dose does not exceed one ninth of the dose required for prophylaxis or treatment of influenza A with said comparative antibody.

37. The antibody for use according to claim 29, wherein the dose does not exceed one tenth of the dose required for prophylaxis or treatment of influenza A with said comparative antibody.

38. The antibody for use according to any one of claims 27-37, wherein the subject to be treated is at immediate risk of influenza A infection.

39. The antibody for use according to any one of claims 27-38, wherein the subject to be treated suffers from an autoimmune disease or an allergy; or is at risk of developing an autoimmune disease or an allergy.

40. A nucleic acid molecule comprising a polynucleotide encoding the antibody of any one of claims 1-26.

41. The nucleic acid molecule of claim 40, wherein the nucleic acid molecule comprises
(i) a polynucleotide comprising a nucleotide sequence as set forth in SEQ ID NO: 12; or a nucleotide sequence having 70% or more identity to SEQ ID NO: 12; and
(ii) a polynucleotide comprising a nucleotide sequence as set forth in SEQ ID NO: 13; or a nucleotide sequence having 70% or more identity to SEQ ID NO: 13.

42. The nucleic acid molecule of claim 40 or 41, wherein the nucleic acid molecule comprises
(i) a polynucleotide comprising a nucleotide sequence as set forth in SEQ ID NO: 14; or a nucleotide sequence having 70% or more identity to SEQ ID NO: 14; and
(ii) a polynucleotide comprising a nucleotide sequence as set forth in SEQ ID NO: 15; or a nucleotide sequence having 70% or more identity to SEQ ID NO: 15.

43. A combination of a first and a second nucleic acid molecule, wherein the first nucleic acid molecule comprises a polynucleotide encoding the heavy chain of the antibody of any one of claims 1-26; and the second nucleic acid molecule comprises a polynucleotide encoding the corresponding light chain of the same antibody.

44. The combination of the first and the second nucleic acid molecule of claim 43, wherein
(i) the first nucleic acid molecule comprises a polynucleotide comprising a nucleotide sequence as set forth in SEQ ID NO: 12; or a nucleotide sequence having 70% or more identity to SEQ ID NO: 12; and
(ii) the second nucleic acid molecule comprises a polynucleotide comprising a nucleotide sequence as set forth in SEQ ID NO: 13; or a nucleotide sequence having 70% or more identity to SEQ ID NO: 13.

45. The combination of the first and the second nucleic acid molecule of claim 43 or 44, wherein
(i) the first nucleic acid molecule comprises a polynucleotide comprising a nucleotide sequence as set forth in SEQ ID NO: 14; or a nucleotide sequence having 70% or more identity to SEQ ID NO: 14; and
(ii) the second nucleic acid molecule comprises a polynucleotide comprising a nucleotide sequence as set forth in SEQ ID NO: 15; or a nucleotide sequence having 70% or more identity to SEQ ID NO: 15.

46. A vector comprising the nucleic acid molecule of any one of claims 40-42.

47. A vector comprising the combination of nucleic acid molecules of any one of claims 43-45.

48. A cell expressing the antibody of any one of claims 1-26, or comprising the vector of claim 46 or 47.

49. A pharmaceutical composition comprising the antibody of any one of claims 1-26, the nucleic acid of any one of claims 40-42, the combination of nucleic acids of any one of claims 43-45, the vector of claim 46 or 47, or the cell of claim 48, and, optionally, a pharmaceutically acceptable diluent or carrier.

50. Use of the antibody of any one of claims 1-26, the nucleic acid of any one of claims 40-42, the combination of nucleic acids of any one of claims 43-45, the vector of claim 46 or 47, the cell of claim 48 or the pharmaceutical composition of claim 49 in the manufacture of a medicament for prophylaxis, treatment or attenuation of influenza A virus infection.

51. The antibody of any one of claims 1-26, the nucleic acid of any one of claims 40-42, the combination of nucleic acids of any one of claims 43-45, the vector of claim 46 or 47, the cell of claim 48 or the pharmaceutical composition of claim 49 for use in prophylaxis or treatment of infection with influenza A virus.

52. The antibody, the nucleic acid, the combination of nucleic acids, the vector, the cell or the pharmaceutical composition for use according to claim 51, wherein the antibody, the nucleic acid, the vector, the cell or the pharmaceutical composition is administered prophylactically.

53. The antibody, the nucleic acid, the combination of nucleic acids, the vector, the cell or the pharmaceutical composition for use according to claim 51 or claim 52, wherein the antibody, the nucleic acid, the vector, the cell or the pharmaceutical composition is administered in combination with an antiviral.

54. The antibody, the nucleic acid, the combination of nucleic acids, the vector, the cell or the pharmaceutical composition for use according to claim 53, wherein the antiviral is selected from neuraminidase inhibitors and influenza polymerase inhibitors.

55. The antibody, the nucleic acid, the combination of nucleic acids, the vector, the cell or the pharmaceutical composition for use according to claim 53 or 54, wherein the antiviral is selected from oseltamivir, zanamivir and baloxavir.

56. The antibody, the nucleic acid, the combination of nucleic acids, the vector, the cell or the pharmaceutical composition for use according to any one of claims 51-55, wherein the subject to be treated suffers from an autoimmune disease or an allergy; or is at risk of developing an autoimmune disease or an allergy.

57. A combination of
(i) the antibody of any one of claims 1-26, and
(ii) an antiviral agent.

58. The combination of claim 57, wherein the antiviral is selected from neuraminidase inhibitors and influenza polymerase inhibitors.

59. The combination of claim 57 or 58, wherein the antiviral is selected from oseltamivir, zanamivir and baloxavir.

60. The combination of any one of claims 57-59 for use in prophylaxis or treatment of infection with influenza A virus.

61. A method of reducing influenza A virus infection, or lowering the risk of influenza A virus infection, comprising: administering to a subject in need thereof, a therapeutically effective amount of the antibody of any one of claims 1-26.

62. The method of claim 61, wherein the antibody is administered prophylactically.

63. The method of claim 61 or 62, wherein the antibody is administered at a dose which does not exceed half of the dose required for prophylaxis or treatment of influenza A with a comparative antibody, which differs from said antibody only in that it does not contain the mutations M428L and N434S in the constant region of the heavy chain.

64. The method of claim 63, wherein the dose does not exceed one third of the dose required for prophylaxis or treatment of influenza A with said comparative antibody.

65. The method of claim 63, wherein the dose does not exceed one fifth of the dose required for prophylaxis or treatment of influenza A with said comparative antibody.

66. The method of any one of claims 61-65, wherein said subject is at immediate risk of influenza A infection.

67. The method of any one of claims 61-66, wherein the antibody is administered in combination with an antiviral.

68. A method of decreasing immunogenicity of an antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; comprising a step of introducing the mutations M428L and N434S in the constant region of the heavy chain of the antibody.
